(19) United States
(12) Patent Application Publication
(10) Pub. No.: US 2025/0356957 A1
(43) Pub. Date: Nov. 20, 2025

(54) EMULATOR OF SUBCUTANEOUS ABSORPTION AND RELEASE

(71) Applicant: UNIVERSITY OF KANSAS, Lawrence, KS (US)

(72) Inventors: Hao LOU, Lawrence, KS (US); Michael HAGEMAN, Lawrence, KS (US)

(21) Appl. No.: 18/869,605

(22) PCT Filed: May 25, 2023
(86) PCT No.: PCT/US2023/067500

§ 371 Date: Nov. 26, 2024

Related U.S. Application Data
(60) Provisional application No. 63/346,228, filed on May 26, 2022.

(51) Int. Cl. G16C 20/30; B01D 61/28; B01D 63/08; G01N 33/15; G16C 20/70

(52) U.S. Cl. CPC G16C 20/30; B01D 61/28; B01D 63/08; G01N 33/15; G16C 20/70; B01D 2313/54

(57) ABSTRACT

A modular in vitro device can be configured as a subcutaneous absorption model. The in vitro device can include a center chamber and a matrix material configured to be included in the center chamber during measurement of absorption of a test agent. A first side chamber is configured to couple with the center chamber, with least one first side opening configured to fluidly couple with the center chamber. A first membrane is configured to be positioned between the center chamber and first side. A second side chamber similar to the first side chamber is provided, with a second membrane configured to be positioned between the center chamber and second side chamber. The center chamber, first side chamber, and second side chamber are configured to be modular for combining with the first membrane and second membrane in a lateral arrangement.

### CROSS-REFERENCE TO RELATED APPLICATIONS

This patent application claims priority to U.S. Provisional Application No. 63/346,228 filed May 26, 2022, which provisional is incorporated herein by specific reference in its entirety.

### BACKGROUND

### Field

The present disclosure relates to devices, systems, and methods of simulating subcutaneous drug action in an in vitro model that mimics subcutaneous conditions, and use of data for modeling and emulating subcutaneous space absorption and release of test agents in an in vivo model.

### Description of Related Art

It is known that the subcutaneous (SC) route of administration can be effective in delivering various types of agents to a subject, such as a human patient or any other animal. The subcutaneous route of administration has demonstrated many advantages in delivering a wide variety of therapeutics, such as small molecules,⁽¹⁾⁽²⁾ peptides,⁽³⁾⁽⁴⁾⁽⁵⁾ proteins (e.g., mAbs),^{(6),(7),(8)} and oligonucleotides (e.g., mRNA, iRNA, and DNA). In order to provide subcutaneous administration to different sites of action, multiple formulations/drug delivery systems are developed, including highly concentrated solutions, semi-aqueous solutions, non-freeze-dried solid formulations, suspensions, liposomes, lipid nanoparticles, drug nano/microparticles, hydrogels, and the like.^{(9),(10),(11),(12)} However, there remains opportunities to improve drug absorption predictability from the subcutaneous injection site.

New modalities may be sought that can overcome prior issues that hamper the accurate prediction of subcutaneous release of a therapeutic from the formulation and absorption from the injection site, which can influence bioavailability and other pharmacokinetic (PK) properties, such as Cₘₐₓ and Tₘₐₓ.^{(13),(14)} Additionally, improvements can be achieved to overcome problems in animal studies that fail to guide human studies due to the lack of translatability for subcutaneous delivery between humans and those commonly-used preclinical animal species.⁽¹⁵⁾

Compared to in vivo models, in vitro models and/or in silico models can have less cost, experimental time, ethical issues, and avoid subject-to-subject variability. Further, in vitro/in silico models can be useful if they can predict drug performance in vivo and even present some level of in-vitro-in-vivo correlations (IVIVC).

The importance of a reliable and robust in vitro system for subcutaneous administration is similar to dissolution apparatuses to oral administration. For oral products, USP apparatuses 1 (basket) and 2 (paddle) are typically used as a routine method in Quality Control (QC) and as a powerful tool for molecule/formulation development in Research & Development (R&D). In addition, researchers can choose more physiologically relevant systems such as USP apparatus 3 (reciprocating cylinder) and gastro-intestinal simulator (GIS).^{(16),(17),(18)} However, for subcutaneous administration there is no standard in vitro system/method/simulated subcutaneous medium.⁽¹⁹⁾

Some studies have been conducted to develop new instruments/systems and apply them in subcutaneous drug formulation dissolution/release tests. These systems include dispersion releaser (DR), subcutaneous injection site simulator (SCISSOR), shake-flask setup, flow-through cell, hydrogel assay in an IVIS® system, UV imaging system, etc.⁽¹⁹⁾ It is noted that dispersion releaser (DR), a modification of a USP apparatus 1 via replacing the basket with a vessel, has presented some capabilities in predicting the in vivo performance of subcutaneous formulations.^{(20),(21),(22)} In addition, SCISSOR is the first commercialized instrument that aims to model the subcutaneous environment and simulate drug migration from the injection site to the absorption site in vivo. A Monte Carlo method has been used to mimic particle movement inside SCISSOR and identified a list of potential critical parameters.⁽²³⁾ Strikingly, SCISSOR has been successfully applied in some research activities associated with molecule screening, formulation development, and bioavailability prediction.^{(24),(25),(26),(27)}

Despite the strengths of DR and SCISSOR, they have some limitations. First, both of them use one donor chamber to represent the subcutaneous site and one acceptor chamber to represent the drug uptake. However, for subcutaneous administration, there exist two drug uptake pathways: (a) the blood pathway, and (b) the lymphatic pathway.⁽²⁸⁾ Hence, the one-acceptor-chamber design cannot investigate two pathways simultaneously. Second, it was impossible to optimize the geometry and hydrodynamics of these in-vitro systems to let them be more similar to the in vivo subcutaneous site.

Thus, there is a need for an in vitro subcutaneous model device that can provide data for computationally modeling subcutaneous administration and absorption of therapeutics in vivo.

### SUMMARY

In some embodiments, a modular in vitro device can be configured as a subcutaneous absorption model. The in vitro device is modular in that the components are modules that can be combined and rearranged, with different center chambers and/or side chambers and/or membranes, which allows for tailored configurations for different subcutaneous environments. The center chamber can be formed by a center chamber body having a first side with at least one first opening and a second side with at least one second opening. A matrix material can be configured to be included in the center chamber during measurement of absorption of a test agent. A first side chamber can be formed by a first side chamber body having a first open side that is configured to couple with the first side of the center chamber. The first open side can have at least one first side opening configured to fluidly couple with the center chamber through the at least one first opening when the first side chamber is mounted to the center chamber. There can be at least one first membrane configured to be positioned between the first side of the center chamber and first open side of the first side chamber to cover the at least one first opening and at least one first side opening. Each first membrane includes a first size exclusion cutoff. A second side chamber can be formed by a second side chamber body having a second open side that is configured to couple with the second side of the center chamber. The second open side can have at least one second side opening configured to fluidly couple with the center chamber through the at least one second opening when the second side chamber is mounted to the center chamber. There can be at least one second membrane configured to be positioned between the second side of the center chamber and second open side of the second side chamber to cover the at least one second opening and at least one second side opening. The second membrane includes a second size exclusion cutoff, which can be less than, the same, or greater than the first size exclusion cutoff. The center chamber, first side chamber, and second side chamber are configured to be modular for combining with the first membrane and second membrane in a lateral arrangement.

In some embodiments, a kit can include the modular in vitro device, which can be configured as a subcutaneous absorption model. The kit may include at least one of: a plurality of different center chamber bodies; a plurality of different matrix materials; a plurality of different first membranes; and a plurality of second first membranes. The first and second side chambers can be fixed in shape and dimensions.

In some embodiments, a system can include the modular in vitro device that can be configured as a subcutaneous absorption model and include at least one fluid circulation system having at least one pump operably coupled with at least one of the center chamber, first side chamber, or second side chamber. The pumps can be connected to one or both side chambers in some aspects, where the center chamber is not connected to any pumps. This allows the side chambers to be sinks for translocation studies.

In some embodiments, the present invention can include a method of modeling subcutaneous absorption. The method can be performed with a system having the modular in vitro device that is configured as a subcutaneous absorption model. The method can include: introducing a test agent in a first amount into the matrix material in the center chamber; allowing the test agent to partition into the first side chamber and/or the second side chamber; measuring an amount of the test agent in at least one of the: (a) center chamber and/or first side chamber and second side chamber; or (b) both the first and second side chambers; determining one or more partition parameters regarding absorption of the test agent into the first side chamber and/or second side chamber; and providing a report having the one or more partition parameters. In some aspects, the method can include: obtaining data of the one or more partition parameters for at least one test agent; modeling the data with a machine learning system; and obtaining a machine learning model of the subcutaneous absorption model. In some aspects, the method can include: obtaining in vitro subcutaneous data with one or more partition parameters of one or more test agents; mapping the one or more partition parameters regarding absorption of the test agent with the in vitro subcutaneous data; and obtaining a correlation model for the subcutaneous absorption model and in vivo data. The correlation model can then allow for obtaining in vivo data that can be used in analyzing the test agent subcutaneous delivery.

In some embodiments, a computer-implemented method can be performed based on data from the modular in vitro device that is configured as a subcutaneous absorption mode. The method can include obtaining partition data of a test agent administered to the in vitro device. The partition data includes a measured amount of the test agent in at least one of the: (a) center chamber and/or first side chamber and second side chamber; or (b) both the first and second side chambers, wherein the in vitro device emulates subcutaneous absorption and release. The method can include creating input vectors based on the partition data of the test agent and inputting the input vectors into a machine learning platform. The method can include generating one or more predicted partition parameters regarding absorption of the test agent from the center chamber into the first side chamber and/or second side chamber by the machine learning platform. The one or more predicted partition parameters are specific to test agent in the model. The method can include preparing a report that includes the one or more predicted partition parameters. In some aspects, the machine learning platform includes a digital model configured to simulate partition parameters in a subcutaneous model of the in vitro device and the digital model is configured to predict in vivo absorption pharmacokinetic properties of the test agent.

In some embodiments, the present invention provides one or more non-transitory computer readable media storing instructions that in response to being executed by one or more processors, cause a computer system to perform operations, the operations comprising a computer-implemented method.

In some embodiments, a computer-implemented method can include: obtaining partition data of a test agent administered to the in vitro device of one of the embodiments, wherein the partition data includes a measured amount of the test agent in at least one of the: (a) center chamber and/or first side chamber and second side chamber; or (b) both the first and second side chambers, wherein the in vitro device emulates subcutaneous absorption and release; creating input vectors based on the partition data of the test agent; inputting the input vectors into a machine learning platform; generating one or more predicted partition parameters regarding absorption of the test agent from the center chamber into the first side chamber and/or second side chamber by the machine learning platform, wherein the one or more predicted partition parameters are specific to the test agent in the subcutaneous model; and preparing a report that includes the one or more predicted partition parameters. In some aspects, the machine learning method performs a Monte Carlo simulation of release of the test agent from the center chamber. In some aspects, the partition data can be based on input factors including matrix concentration, test agent injection volume, test agent injection position, and combinations thereof. In some aspects, the machine learning platform models a relationship between the input factors and output responses based on a subcutaneous model of the in vitro device.

In some embodiments, the present invention includes a computer system that has one or more processors and one or more non-transitory computer readable media storing instructions that in response to being executed by the one or more processors, cause the computer system to perform operations of a computer-implemented method.

In some embodiments, a computer-implemented method can include: obtaining partition data of a test agent administered to the in vitro device of one of the embodiments, wherein the partition data includes a measured amount of the test agent in at least one of the: (a) center chamber and/or first side chamber and second side chamber; or (b) both the first and second side chambers, wherein the in vitro device emulates subcutaneous absorption and release; creating input vectors based on the partition data of the test agent; inputting the input vectors into a machine learning platform; generating one or more predicted partition parameters regarding absorption of the test agent from the center chamber into the first side chamber and/or second side chamber by the machine learning platform, wherein the one or more predicted partition parameters are specific to test agent in the model; and preparing a report that includes the one or more predicted partition parameters. In some aspects, the partition data can be based on input factors including matrix concentration, test agent injection volume, test agent injection position, and combinations thereof. In some aspects, the machine learning platform models a relationship between the input factors and output responses based on a subcutaneous model of the in vitro device, and the machine learning method performs a Monte Carlo simulation of release of the test agent from the center chamber. The result is a computer simulation that models in vivo subcutaneous administration of a test agent.

In some embodiments, a computer-implemented method can include: obtaining partition data of a test agent administered to the in vitro device of one of the embodiments, wherein the partition data includes a measured amount of the test agent in at least one of the: (a) center chamber and/or first side chamber and second side chamber; or (b) both the first and second side chambers, wherein the in vitro device emulates subcutaneous absorption and release; modeling the partition data with a digital model of the subcutaneous model of the in vitro device; generating one or more predicted partition parameters regarding absorption of the test agent from the center chamber into the first side chamber and/or second side chamber; and preparing a report that includes the one or more predicted partition parameters. In some aspects, the digital model is an in vitro model. In some aspects, the digital model is an in vivo model.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIGS. 1A-1B include schematic representations of an in vitro subcutaneous model device connected to a fluid flow control system, which simulates the in vivo subcutaneous environment for test agent administration.

FIGS. 2A-2E include schematic representations of a center chamber module that mimics the subcutaneous region that receives the drug administration.

FIGS. 3A-3F include graphs that include release profiles of 18 DoE runs using 10 mg/mL acetaminophen solution: (3A) release medium: 2.5 mg/mL HA solution; Injection position: 0.5 cm to the membrane; three injection volume: 0.25 mL, 0.5 mL, 1 mL; (3B) release medium: 5 mg/mL HA solution; Injection position: 0.5 cm to the membrane; three injection volume: 0.25 mL, 0.5 mL, 1 mL; (3C) release medium: 10 mg/mL HA solution; Injection position: 0.5 cm to the membrane; three injection volume: 0.25 mL, 0.5 mL, 1 mL; (3D) release medium: 2.5 mg/mL HA solution; Injection position: 0.2 cm to the membrane; three injection volume: 0.25 mL, 0.5 mL, 1 mL; (3E) release medium: 5 mg/mL HA solution; Injection position: 0.2 cm to the membrane; three injection volume: 0.25 mL, 0.5 mL, 1 mL; (3F) release medium: 10 mg/mL HA solution; Injection position: 0.2 cm to the membrane; three injection volume: 0.25 mL, 0.5 mL, 1 mL.

FIG. 4 includes a graph that shows the prediction profiler plots of release fraction (%) at 2-hr versus three factors (HA concentration, injection volume, injection position to the membrane.

FIG. 5 includes a graph that shows the Monte Carlo simulation of drug release from the subcutaneous (center) chamber based on: the insertion of 256 particles into the chamber; the insertion of 512 particles into the chamber; the insertion of 960 particles into the chamber; particle migration inside the chamber as a function of arbitrary time unit (MCS); the correlation between the simulation data (q: 0.45) and the actual data (release medium: 5 mg/mL HA solution); the correlation between the simulation data (q: 0.8) and the actual data (release medium: 5 mg/mL HA solution). The release fraction versus actual time shows the Monte Carlo Simulation for 5 mg/mL HA at the different injection volumes.

FIG. 6A shows the two-compartment PK model for the SC administration of griseofulvin suspension.

FIG. 6B includes a graph that shows the In-Vitro-In-Vivo Correlation (IVIVC) model development for the in vitro permeated release profile (mg) over time obtained from the in vitro subcutaneous model device (ESCAR).

FIG. 6C includes a graph that shows the In-Vitro-In-Vivo Correlation (IVIVC) model development for the plasma concentration (micro molar) over time obtained from the in vitro subcutaneous model device (ESCAR).

FIG. 7 shows the BSA release profiles after the 0.5-mL injection of BSA solution (30 mg/mL) into the subcutaneous (center) chamber filled with PBS or HA solution.

FIG. 8 shows a three-dimensional embodiment of the in vitro subcutaneous model device (ESCAR) having a center chamber and two side chambers.

FIG. 9 shows a three-dimensional embodiment of the in vitro subcutaneous model device (ESCAR) having a main chamber and one side chamber.

FIG. 10 includes a schematic diagram of a computing device that can be used in the present invention.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention provides an in vitro assay device that is configured to simulate a subcutaneous environment with a center/middle chamber and two side chambers that are each separated from the center/middle chamber by a membrane. The in vitro subcutaneous model device can be configured to be modular in that different center chambers, different matrix materials, different side chambers and different membranes can be used to modulate the translocation of test agents between the different chambers. This allows for the in vitro subcutaneous model device to be tailored to mimic a physiological condition of a subcutaneous location that receives an administered test agent. The subcutaneous model device is also tailored so that the test agent translocation data can be used for modeling the subcutaneous administration of the test agent, and translocation of the test agent from the site of administration to a physiological position.

The in vitro subcutaneous model device described herein can be referred to as ESCAR (e.g., which stands for Emulator of SC Absorption and Release). The ESCAR device can be configured with modular components to be used for simulating therapeutic subcutaneous administration and release thereof by absorption into regions outside of the administration site. The ESCAR device allows for the in vitro studying of the drug-like action of the test agent in the release and absorption inside the subcutaneous (SC) space, which is comparable to the conditions that are found in vivo for subcutaneous administration.

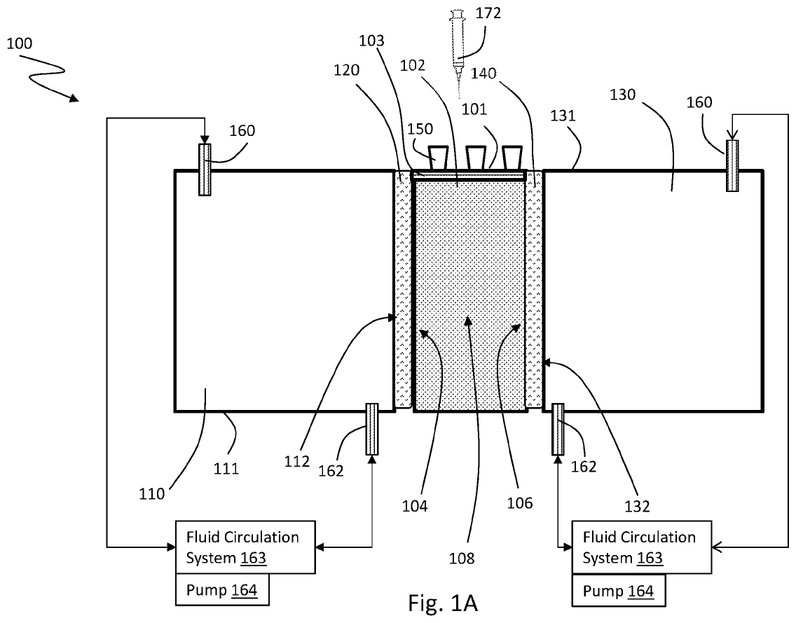

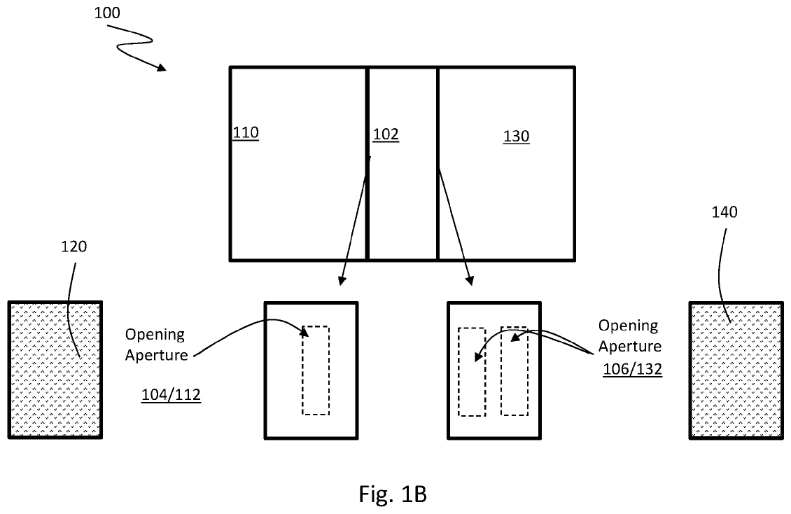

FIGS. 1A-1B show the in vitro device 100 configured as an in vitro subcutaneous absorption model having the center chamber 102 with the first side chamber 110 on one side and a second side chamber 130 on the other side. As shown, the subcutaneous absorption model 100 can include the center chamber 102 formed by a center chamber body 101 having a first side with at least one first opening 104 and a second side with at least one second opening 106. The first side is opposite of the second side. However, it is possible to put the second side with the at least one second opening 106 at an angle relative to the first side with the at least one first opening 104. Therefore, the relative angle between each first opening 104 and each second opening 106 can range from parallel to 90 degrees. As shown herein, the parallel embodiment is used to exemplify the device 100.

During use, a matrix material 108 is in the center chamber 102. The matrix material 108 can be included in the center chamber during manufacturing or introduced at some point before performance of the in vitro subcutaneous absorption assay. The matrix material 108 can include a polysaccharide material or other natural or synthetic polymer matrix that can simulate the subcutaneous injection administration and translocation into other physiological regions. The test agent can be injected into a position in the matrix material, which then allows for the translocation of the test agent therethrough until reaching a boundary membrane 120, 140.

The device 100 includes a first side chamber 110 formed by a first side chamber body 111 having a first open side that is configured to couple with the first side of the center chamber 102. The first open side of the first side chamber 110 has at least one first side opening 112 that is configured (e.g., dimensioned, positioned, oriented, etc.) to fluidly couple the first side chamber 110 with the center chamber 102 through the at least one first opening 104. The first side chamber 110 and center chamber 102 can be fluidly coupled when the first side chamber 110 is mounted onto to the center chamber 102 such that the first opening 104 connects with the first side opening 112.

The membranes 120, 140 can include a first membrane 120 that is configured to be positioned between the first side of the center chamber 102 and the first open side of the first side chamber 110 to cover the at least one first opening 104 and/or first side opening 112. That is, the membrane 120 provides a barrier to translocation of a test agent from the center chamber 102 into the first side chamber 110 or otherwise therebetween. The membrane 120 may be held in a membrane frame, or fit into a membrane-retaining region of either the central chamber 102 or first side chamber 110. In some aspects, the membrane 120 can be pressed between the body 101 and body 111. In some aspects, the first membrane 120 can include a first size exclusion cutoff.

The device 100 includes a second side chamber 130 formed by a second side chamber body 131 having a second open side that is configured to couple with the second side of the center chamber 102. The second open side of the second side chamber 130 can have at least one second side opening 132 that is configured to fluidly couple with the center chamber 102 through the at least one second opening 106. The center chamber 102 can be fluidly coupled with the second side chamber 130 when the second side chamber 130 is mounted to the center chamber 102 such that each second opening 106 connects with the second side opening 132.

The membranes, 120, 140 can include a second membrane 140 that is configured to be positioned between the second side of the center chamber 102 and the second open side of the second side chamber 130 to cover the at least one second opening 106 and/or second side opening 132. That is, the second membrane 140 provides a barrier to translocation of a test agent from the center chamber 102 into the second side chamber 130 or otherwise therebetween. The membrane 140 may be held in a membrane frame, or fit into a membrane-retaining region of either the central chamber 102 or second side chamber 130. In some aspects, the membrane 140 can be pressed between the body 101 and body 131. In some aspects, the second membrane 140 includes a second size exclusion cutoff.

As shown, the center chamber 102, first side chamber 110, and second side chamber 130 are configured to be modular for combining with the first membrane 120 and second membrane 140, which can be in a lateral arrangement.

In some embodiments, the ESCAR device includes three main chambers separated by membranes. The middle chamber (i.e., center chamber 102) is the subcutaneous chamber, which emulates multiple actions occurring inside the subcutaneous injection site. The subcutaneous chamber can be filled with a simulated subcutaneous medium (e.g., matrix material 108). The center chamber 102 may be referred to as the middle chamber, subcutaneous chamber, or other similar term.

In some embodiments of the device 100 of FIG. 1, the ESCAR device can include three compartments: the “subcutaneous” chamber (102), the “blood circulation” chamber (110), and the “lymphatic circulation” chamber (130) or second “blood circulation” chamber (130). The subcutaneous chamber, representing the subcutaneous site, can be a rectangular cuboid or any other geometric shape with two open surfaces or faces at opposite sides. The top side of the subcutaneous chamber can have either a ceiling that can be integrated with the whole chamber or configured as a lid, or have an open window that can be sealed by a membrane during experiments, or any combination thereof.

In some embodiments, injection ports 150 are built on top of the subcutaneous chamber (102) in a precise location to ensure the accurate injection of drug formulations. The injection ports 150 can be provided in any number and in any arrangement, such as from at least one injection port 150 to any number that fit. This can allow for tailoring the injection site into the matrix material 108 in the center chamber 102. The injection ports can be apertures or holes, or can include a port member that facilitates injection.

In some embodiments, one of the injection ports 150 can be configured as an optical viewing port. This allows for an optical imaging device to be optically coupled, such as mounted above or inserted into the center chamber 102. For example, a catheter-like imaging device can be inserted into the matrix material, or a top-mounted video camera can be installed to visually track the test agents. Accordingly, the test agents can include markers that are visually identifiable, such as fluorescent labels.

In some embodiments, there can be different designs of the subcutaneous chamber, varying with chamber volume, shape, and contact surface area/shape for the membranes as well as the openings in the center chamber and/or the side chambers. The two side chambers can be configured to represent the (1) the blood circulation chamber and the lymphatic circulation chamber, when considering both the lymphatic and blood absorption pathway simultaneously; or (2) both can be used as blood circulation chambers, while the lymphatic absorption pathway is not considered, or considered to be negligible. The blood/lymphatic circulation chambers have various sizes to accommodate emulations with different formulations/doses. At the contact surface of two side chambers with the center chamber, the membrane interface can be configured to be representative of either lymphatic or blood limiting membranes. The device can be assembled with the partition membranes in order to control the test agent (e.g., molecule, protein, therapeutic, etc.) migration from the center chamber, through the membrane and into the side chambers. The three chambers are aligned horizontally and can be coupled together by any coupling means. Coupling of chambers together can include custom-coupling features that interlock and hold the adjacent chamber bodies together, or the coupling can be achieved by tightened and adjusting knobs, clamps, fasteners, bolts, screws, adhesive, or any other.

The device 100 can be configured such that the center chamber 102 includes: a top cover 103 that is a solid sheet with an inlet port 150; or a simulated skin layer, which can optionally be parafilm.

The side chambers 110, 130 can include inlet ports 160 and exit ports 162 that are coupled with fluid circulation systems 163 that include a pump 164 and optionally temperature regulators, such as heater, cooler, filers, or the like.

An injector 172, such as a syringe, can be used to inject the test agent into the center chamber 102. The ports 150 can be configured with a membrane for receiving injection via needle therethrough.

FIG. 1B shows the first aperture opening 104/112 that is the interface between the center chamber 102 and the first chamber 110 and the second aperture opening 106/132 that is the interface between the center chamber 102 and the second chamber 130. The first aperture opening 104/112 can be defined by either the center chamber body 101 or the first chamber body 111, or some other separate member. In any embodiment, the aperture opening 104/112 defines the space for test agents to translocate between the center chamber 102 and the first side chamber 110. Similarly, the second aperture opening 106/132 can be defined by either the center chamber body 101 or the second chamber body 131, or some other separate member. In any embodiment, the second aperture opening 106/132 defines the space for test agents to translocate between the center chamber 102 and the second side chamber 130. Accordingly, the dimensions of the cross-sectional area of the first aperture opening 104/112 and/or the second aperture opening 106/132 can be modulated in order to provide a desired translocation potential. Also, the number of aperture openings in an interface between the center chamber 102 and one of the side chambers 110, 130 may be varied from 1, 2, 3, 4, or any number. In some aspects, there can be two aperture openings at an interface that are spaced apart from each other. Therefore, the shape, dimension, and number of aperture openings in the interface can be modulated in order to tune the translocation kinetics to simulate the test agent in an in vitro model to provide data for a computational in vivo model.

The convection within the subcutaneous compartment (center chamber 102) can be integrated into the system by connecting external liquid flow via the fluid circulation system 163 into the center chamber 102. The device 100 can be fabricated with traditional casting technology or using a 3D printer. While the body parts of the device 100 can be made of any material, an example is ABS (acrylonitrile butadiene styrene). Other materials and fabrication techniques can be chosen for various reasons or for tailoring for different applications.

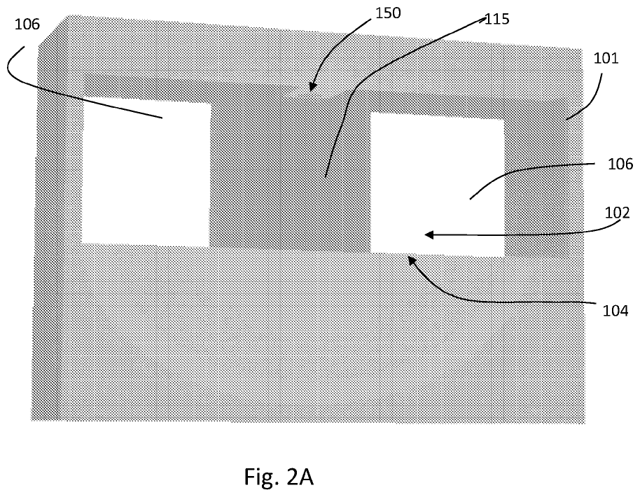

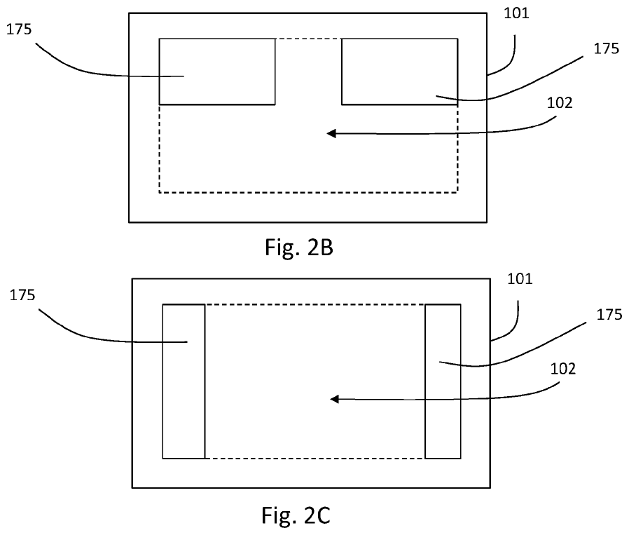

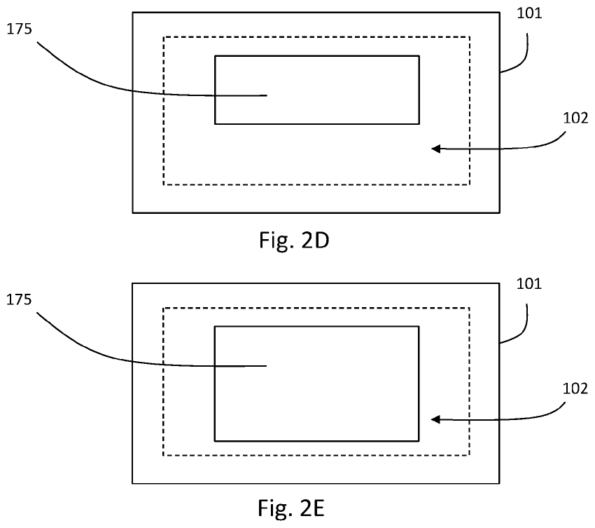

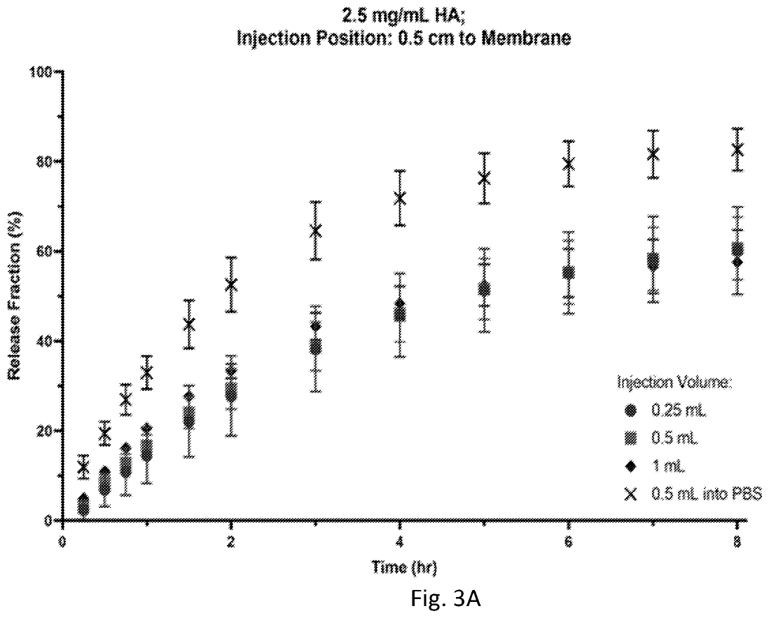

FIG. 2A shows the center chamber body 101 defining the center chamber 102, where the first opening 104 has a cross-sectional profile that matches the cross-sectional profile of the center chamber 102. The center chamber body 101 is shown to define two second openings 106 across from the first opening 104. The two second openings 106 are separated by a barrier wall 115. The width of the barrier wall 115 can vary along with the width of the second openings.

FIGS. 2B-2C shows the center chamber body 101 defining at least one opening 175 (e.g., first opening 104 or second opening 106; apertures) with the barrier wall 115 defining the at least one opening 174. The FIGS. 2B-2C show different embodiments of the center chamber body and openings 175 to the center chamber 102. As shown, there can be any number and arrangement of openings 175 (apertures) in the barrier wall, where two spaced apart openings 175 is specifically exemplified, in horizontal (FIG. 2B) and vertical (FIG. 2C) orientations (e.g., Version 1). Also, a single opening 175 of close to the same cross-sectional profile (FIG. 2E) of the center chamber 102 or a different (FIG. 2D) size (e.g., Version 2). However, in one embodiment, the invention includes at least one of the interfaces of the body of FIG. 2B or 2C (human) and one of the bodies of FIG. 2D or 2E (rat). In some embodiments, the interface mimicking the lymphatic system can be wide open without restriction by being the same cross-sectional profile as the center chamber.

FIGS. 2A-2E show an embodiment of the interface between the center chamber 102 and the first side chamber 110 and/or the second side chamber 130. The interface between the center chamber 102 and the second side chamber 130 can be the same or different, which may be completely open without any barrier.

FIGS. 2B and 2C show barrier wall 115 between the pair of opening apertures 175. The body that provides the first interface can be the center chamber body 101 and/or the first side chamber body 111. The second interface can be the center chamber body 101 or second side chamber body 131. That is, the openings can be formed into the center chamber body and/or the first side chamber body and/or they openings can be formed into a separate member that can be located between the center chamber body and first side chamber that includes the interface.

Another embodiment shows a single open aperture 175 as in FIGS. 2D and 2E. These can be used for rat models or other leaky lymphatic or blood characteristics that can be modeled.

The area of the aperture 175 of each opening can be added to determine the full aperture area for the first opening 104, second opening 106, first side opening 112 and second side opening 132. This information can be used in the computation of the data to mimic in vivo conditions.

In FIGS. 2B-2E, the dashed line shows the cross-sectional profile of the center chamber 102.

In some embodiments, the present in vitro ESCAR device provides the following advantages. For some prior devices, only one receiver chamber is available for simulating drug absorption. On the other hand, the ESCAR device has two receiver chambers that are designed to represent capillary blood and lymphatic absorption. For some prior devices, the membrane and subcutaneous chamber are glued together, and researchers have no flexibility to evaluate different membranes of their choice. For the in vitro ESCAR device, the membranes are detachable, and the three chambers are separate modular parts, which allows researchers to freely assemble tailored configurations, such as with different types of membranes to conduct studies for different test agents.

In some embodiments, the in vitro subcutaneous device can be used in various types of in vitro experiments, which are designed to obtain data for use in modeling the corresponding in vivo subcutaneous administration. For example, the experimental design can include: (1) drug molecule screening, such as for a small molecule, peptide, oligonucleotide, antibody, ligand, biological molecule, or the like; (2) subcutaneous formulation development and optimization; (3) IVIVC (In-Vitro-In-Vivo-Correlation) by using the ESCAR data in computing models that can be used to predict in-vivo drug absorption for modeling of pharmacokinetic (PK) properties, such as bioavailability, lymphatic uptake profile, plasma PK profile); (4) IVIVC: using ESCAR data to predict in-vivo subcutaneous injection-related variables and activities; (5) exploration of potential events occurring in an subcutaneous space, such as drug aggregation, degradation, and drug-hyaluronic acid interaction; (6) the ESCAR device can be integrated with other techniques such as 3D cell culture and on-line analysis more easily; and (7) various other applications.

In some embodiments, the in vitro device system (ESCAR) can be used to assess the subcutaneous administration of test agents, such as small molecules and large molecules. In some aspects, a hyaluronic acid (HA) solution can be used to simulate the subcutaneous extracellular matrix (ECM). Further, if drug hydrophobicity and adipose tissue/skin lipid are taken into consideration, an O/W emulsion containing lecithin (e.g., oil-like phase) and HA/PBS (aqueous phase) can be used as the simulated subcutaneous medium. Accordingly, the matrix material can be tailored for the intended use and assay conditions. A series of process parameters including HA concentration, injection volume, and injection position (e.g., needle tip position) can be systemically investigated by a Design-of-Experiment (DoE) study. Further, an IVIVC model can be developed with the application of ESCAR data.

The open surfaces at the front and back sides between the center chamber and two side chambers represent two drug uptake pathways: (a) the blood pathway (e.g., first side chamber 110), and (b) the lymphatic pathway (e.g., second side chamber 130) or a second blood pathway. Inside the subcutaneous region, there is an extensive distribution of blood microcirculation that is mainly organized and controlled by horizontal plexuses at the dermal-subcutaneous junctions, as well as many capillaries extending into deep adipose tissue.^{(29),(30)} On the contrary, there was less distribution of lymphatic vessels, which were mainly relatively large lymphatic vessels, but were seldom microcirculatory lymphatic vessels.^{(31),(32)} The center chamber can be designed to reflect this unequal distribution: (a) the subcutaneous/blood circulation interface (the front side) had an open surface with 3 cm in length×1 cm in height, and (b) the subcutaneous/lymphatic circulation interface (the back side, first interface 200) had a 2-mm thickness slab at the center (barrier wall 115), and two open slits (175; 0.1 cm in length×1 cm in height) at the left and right ends (FIG. 2C; Version 1). So, “blood” uptake had a larger surface area by being completely open and a shorter distance from the injection site compared to “lymphatic” uptake having the smaller aperture area.

The center chamber can be optimized to test small molecules with a focus on blood absorption. However, to evaluate large molecules, a larger surface area at the back side can be used in order to obtain faster drug release and shorter experimental time. Thus, both the apertures at the interfaces of the center chamber with the side chambers can be configured and optimized to provide physiologically relevant data.

Further, lymphatic capillaries can be simulated and can be configured more “open” compared to blood capillaries. The outer walls of the lymphatic capillary simulation can be composed of a single layer of loosely adherent and overlapped endothelial cells. The “cleft-like” intercellular junctions (junction size: in the range of 15 nm and 100 nm to even several microns) allowed fluid, as well as macromolecules/colloids in the fluid, freely entering the lymphatics.^{(33),(13),(34),(35)} These intercellular junctions can be emulated by a membrane (e.g., 120/140) with pores. For instance, a SpectraPor® dialysis membrane with 300 kDa MWCO can be used; however, larger pores may be used for other embodiments. Unlike lymphatic capillary walls, tight inter-endothelial junctions (e.g., adherents junctions and tight junctions) can be present on blood capillary walls, which restricted the paracellular transport of molecules with the size larger than 3 nm, although some macromolecules such as albumin, hormones, insulin, etc. could still cross endothelial cells via transcellular or transcytotic pathways.^{(33),(13),(34),(35),(36)} Previous studies reported that a series of proteins followed a trend that the extent of lymphatic uptake increased with molecular weight, e.g., insulin (MW: 5.8 kDa),⁽³⁷⁾ cytochrome c (MW: 12.3 kDa),⁽³⁸⁾ human growth hormone (MW: 22 kDa),⁽³⁹⁾ rHuEPO (MW: 30.4 kDa),⁽⁴⁰⁾ and darbepoetin alfa (MW: 37.0 kDa).^{(41),(42)} For example, blood uptake was the primary pathway for small molecules, and lymphatic uptake was the main pathway for large molecules. In the present ESCAR design, a SpectraPor® dialysis membrane (MWCO: 50 kDa) was used as an MW cutoff for “blood” uptake. Therefore, the first membrane 120 and second membrane 140 can be configured appropriately.

To emulate the infinite sink condition after drug uptake, both the “blood circulation” (110) and the “lymphatic circulation” chambers (130) were larger than 75 mL, which was at least 20 orders of magnitude to the volume of the center chamber (102). However, it is possible that the side chambers may only be 10 orders of magnitude larger than the center chamber. In addition, optimization can be aimed to emulate the unidirectional fluid flow through subcutaneous interstitium to lymphatic capillaries in vivo. According to the Starling theory, this flow was driven by the capillary hydrostatic pressure between arteriole and interstitium, as well as the interstitial colloid osmotic pressure.^{(43),(44)} The flow rate from the interstitium into the lymphatic system typically ranged from 0.2 to 1 μm/s.⁽⁴⁵⁾ It was reported that the migration of large molecules inside the subcutaneous ECM was significantly impacted by convection, while the migration of small molecules was mainly controlled by diffusion.⁽⁷⁾ In ESCAR, convection could be generated by feeding a liquid flow into the center subcutaneous chamber 120 using a syringe pump 172.

Furthermore, the subcutaneous chamber can be optimized by design to represent the subcutaneous sites of rats. It was reported that for rats, the extent of lymphatic uptake was very low for small molecules, and even for some large molecules,⁽⁴⁶⁾ therefore both sides of the subcutaneous center chamber 102 were designed for “blood” uptake (e.g., wide open aperture at interface) to mimic rats. Also, different configurations can have a larger chamber volume and interface surface area, to mimic rats that have more loose subcutaneous connective tissue compared to humans, and therefore injected formulation would spread more widely and rapidly in rat's subcutaneous site.⁽⁴⁷⁾

The in vitro model device can be configured such that the matrix material includes: a hydrophilic polysaccharide material, optionally a glycosaminoglycan (GAG), optionally a negatively charged polysaccharide material, optionally hyaluronic acid or hyaluronate; or a hydrophobic material within the hydrophilic polysaccharide material, wherein the hydrophobic material is optionally a lipid, optionally a lecithin.

The in vitro model device can be configured such that the model includes at least one of: the center chamber body having at least one port 150 that is optionally adapted to be coupled to a fluid circulation system or a sample acquisition unit; the first side chamber body having at least one port 160/162 that is optionally adapted to be coupled to the fluid circulation system or a sample acquisition unit; or the second side chamber body having at least one port 160/162 adapted to be coupled to the fluid circulation system. See FIG. 1A.

The in vitro model device can be configured such that at least one of: the first side chamber is configured as a blood absorption chamber and the second side chamber is configured as a lymphatic absorption chamber, wherein the second side of the center chamber includes the two spaced apart second openings that have a combined open area less than an open area of the one first opening; or the first side chamber is configured as a first blood absorption chamber and the second side chamber is configured as a second blood absorption chamber, wherein the second side of the center chamber includes the one second opening that has an open area that is about the same as an open area of the one first opening.

A kit can include: the in vitro model device of one of the model embodiments, comprising at least one of: a plurality of different center chamber bodies; a plurality of different matrix materials; a plurality of different first membranes; and a plurality of second first membranes.

A system can include: the in vitro model device of one of the model embodiments; and at least one fluid circulation system having at least one pump (or fluid circulation system) operably coupled with at least one of the center chambers, first side chamber, or second side chamber. The system can include at least one analytical component configured to obtain data of diffusion of a test agent (e.g., molecule) from the center chamber to at least one of the first side chamber or second side chamber. The system can include at least one test agent in the matrix material in the center chamber, wherein the test agent diffuses/absorbs into at least one of the first side chamber or second side chamber.

In this study, a prototype of an in vitro system ESCAR was developed to emulate the in vivo subcutaneous environment. ESCAR showed its potential uses in the assessment of different subcutaneous formulations (e.g., solution and suspension) and small molecule drugs (e.g., hydrophilic molecule: acetaminophen; hydrophobic molecule: griseofulvin). From a factor screening study, it was found that drug release from the subcutaneous chamber was significantly affected by HA concentration rather than injection volume and injection position. Further, a Monte Carlo simulation-based method was developed to model the drug release in the high HA (e.g., 5 or 10 mg/mL) medium, and the simulation data were in accordance with the experimental data. An IVIVC model was successfully developed. This established IVIVC model demonstrated that the ESCAR device had important implications in subcutaneous drug product development and bioequivalence studies.

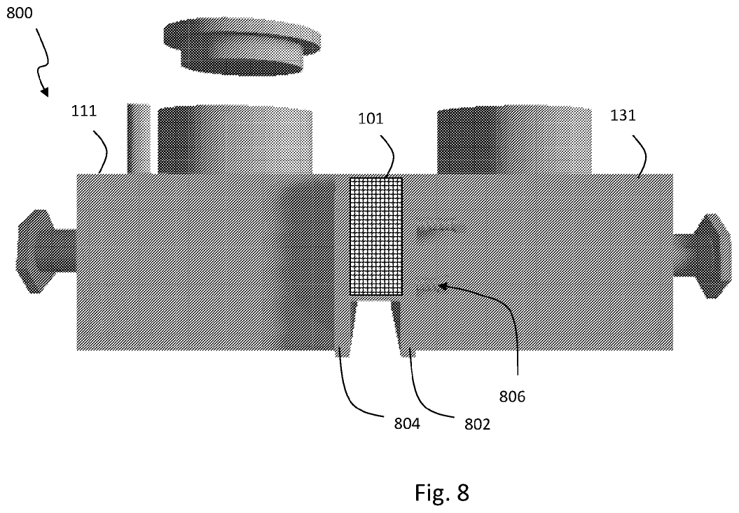

FIG. 8 shows a 3D image of the in vitro subcutaneous device 800 having the center chamber body 101, first side chamber body 111 and second side chamber body 131 coupled together with the membranes therein. The first side chamber body 111 includes a first flange 804 and the second side chamber body 111 includes a second flange 802 with threaded holes that receive threaded fastener 806 (bolt) therethrough to couple the bodies together.

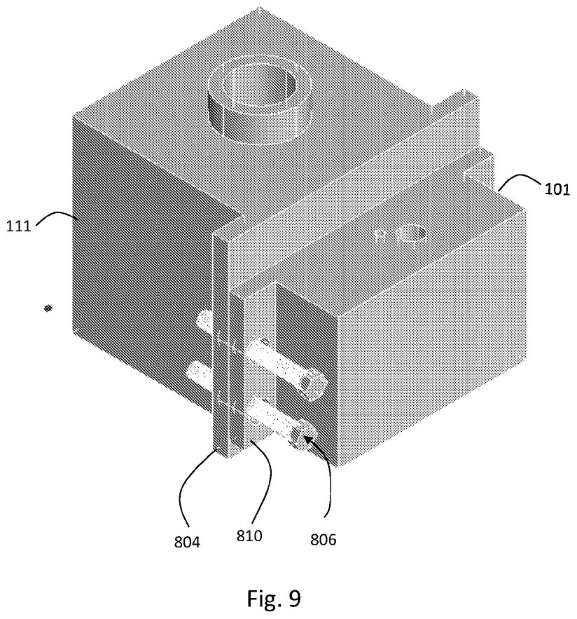

FIG. 9 shows a 3D image of the in vitro subcutaneous device 900 having the center chamber body 101 and only the first side chamber body 111 coupled together with the membranes therein. The first side chamber body 111 includes a first flange 804 and the center chamber body 101 (e.g., main chamber body) includes a second flange 810 with threaded holes that receive threaded fastener 806 (bolt) therethrough to couple the bodies together.

A reliable in vitro system can support and guide the development of subcutaneous (SC) drug products. Although several in vitro systems have been developed, they have some limitations, which may hinder them from getting more engaged in subcutaneous drug product development. This study sought to develop a novel in vitro system, namely Emulator of subcutaneous Absorption and Release (ESCAR), to better emulate the in vivo subcutaneous environment and predict the fate of drugs in subcutaneous delivery. ESCAR was fabricated using the 3D printing technique and was used to evaluate different molecules (hydrophilic and hydrophobic small molecules) and formulations (solution and suspension). A DoE factor screening study was conducted to identify critical parameter(s). An in-vitro-in-vivo correlation (IVIVC) study was developed to explore the feasibility of applying ESCAR in formulation development and bioequivalence studies. The results of the factor screening study suggested that hyaluronic acid (HA) concentration was a critical parameter for drug release, whereas the influence of injection volume and injection position within the device was not substantial. Further, drug release from the subcutaneous chamber to the acceptor chamber could be modeled by a variety of methods, including polynomial equations, machine learning methods, and Monte Carlo simulation-based methods. The developed LEVEL-A IVIVC model demonstrated that the in vivo PK profile could be correlated to the in vitro release profile. Therefore, using this model, for new formulations, only in vitro studies need to be conducted in ESCAR, and in vivo studies might be waived. In conclusion, ESCAR had important implications in research & development and quality control of subcutaneous drug products.

### Embodiments

In some embodiments, a modular subcutaneous absorption model can include a center chamber formed by a center chamber body having a first side with at least one first opening and a second side with at least one second opening. The first side is opposite of the second side. A matrix material is provided that is configured to be included in the center chamber during measurement of absorption of a test agent. The matrix material can include a polysaccharide material or other matrix medium. A first side chamber is provided that is formed by a first side chamber body having a first open side that is configured to couple with the first side of the center chamber. The first open side has at least one first side opening configured to fluidly couple with the center chamber through the at least one first opening when the first side chamber is mounted to the center chamber. A first membrane is provided that is configured to be positioned between the first side of the center chamber and first open side of the first side chamber to cover the at least one first side opening. The first membrane includes a first size exclusion cutoff. A second side chamber is provided that is formed by a second side chamber body having a second open side that is configured to couple with the second side of the center chamber. The second open side has at least one second side opening configured to fluidly couple with the center chamber through the at least one second opening when the second side chamber is mounted to the center chamber. A second membrane is provided that is configured to be positioned between the second side of the center chamber and second open side of the second side chamber to cover the at least one second side opening. The second membrane includes a second size exclusion cutoff. The center chamber, first side chamber, and second side chamber are configured to be modular for combining with the first membrane and second membrane in a lateral arrangement.

In some embodiments the center chamber, first side chamber, and second side chamber are coupled together and combined with the first membrane and second membrane in the lateral arrangement, optionally the first side chamber and/or second side chamber includes an absorbing medium.

In some embodiments, the center chamber includes: a top cover that is a solid sheet with an inlet port; or a simulated skin layer, optionally parafilm.

In some embodiments, the matrix material includes: (a) a hydrophilic polysaccharide material, optionally a glycosaminoglycan (GAG), optionally a negatively charged polysaccharide material, optionally hyaluronic acid or hyaluronate; or (b) a hydrophobic material within the hydrophilic polysaccharide material, wherein the hydrophobic material is optionally a lipid, optionally a lecithin.

In some embodiments, the center chamber body includes one of: (a) the first side includes one first opening and a second side includes two second openings that are spaced apart from each other, wherein the two second openings have a combined open area that is smaller than an open area of the one first opening; or (b) the first side includes one first opening and a second side includes one second opening, wherein the one second opening has an open area that is equal to or smaller than an open area of the one first opening.

In some embodiments, the combined open area of the two second openings is less than 90%, 80%, 70%, 60%, 50%, 40%, 30%, 20%, 10%, 5%, or 1% of the open area of the at least one first opening.

In some embodiments, the first side chamber and second side chamber each have a chamber volume larger than a chamber volume of the center chamber, optionally, 1.5 times larger, 2 times larger, 2.5 times larger, 3 times larger, 3.5 times larger, 4 times larger, 4.5 times larger, 5 times larger, 10 times larger, 20 times larger, 50 times larger, or 100 times larger.

In some embodiments, the center chamber can have a volume of about 1 mL to about 50 mL, or about 5 mL to about 25 mL, or about 7 mL to about 15 mL, or about 10 mL.

In some embodiments, the membranes can be configured as one of the following: the first size exclusion cutoff is less than or about 100 kDa and the second size exclusion cutoff is greater than or about 100 kDA; the first size exclusion cutoff is less than or about 75 kDa and the second size exclusion cutoff is greater than or about 200 kDA; or the first size exclusion cutoff is less than or about 50 kDa and the second size exclusion cutoff is greater than or about 300 kDA.

In some embodiments, the device can be configured with at least one of: the center chamber body having at least one port that is optionally adapted to be coupled to a fluid circulation system or a sample acquisition unit; the first side chamber body having at least one port that is optionally adapted to be coupled to the fluid circulation system or a sample acquisition unit; or the second side chamber body having at least one port adapted to be coupled to the fluid circulation system.

In some embodiments, a system or kit can include a plurality of different center chamber bodies, each center chamber body having a unique open area of the at least one second side opening. The first and second side chambers may be present in standard configurations, or different versions may be provided in the system or kit.

In some embodiments, the device can include at least one of: the first side chamber is configured as a blood absorption chamber and the second side chamber is configured as a lymph absorption chamber, wherein the second side of the center chamber includes the two spaced apart second openings that have a combined open area less than an open area of the one first opening; or the first side chamber is configured as a first blood absorption chamber and the second side chamber is configured as a second blood absorption chamber, wherein the second side of the center chamber includes the one second opening that has an open area this about the same as an open area of the one first opening.

In some embodiments, a kit can include: the in vitro subcutaneous model device of one of the embodiments described herein, comprising at least one of: a plurality of different center chamber bodies; a plurality of different matrix materials; a plurality of different first membranes; and a plurality of second first membranes. The first side chamber and second side chambers can be fixed, and single embodiments thereof provided.

In some embodiments, a system can include the in vitro subcutaneous model device of one of the embodiments; and at least one fluid circulation system having at least one pump operably coupled with at least one of the center chamber, first side chamber, or second side chamber. In some aspects, the system can include at least one analytical component configured to obtain data of absorption of a molecule from the center chamber to at least one of the first side chamber or second side chamber. In some aspects, the system can include at least one test reagent in the matrix material in the center chamber, wherein the test reagent absorbs into at least one of the first side chamber or second side chamber.

In some embodiments, a method of modeling subcutaneous absorption can include: providing the in vitro subcutaneous model device of one of the embodiments; introducing a test agent in a first amount into the matrix material in the center chamber; allowing the test agent to partition into the first side chamber and/or the second side chamber; measuring an amount of the test agent in at least one of the: (a) center chamber and/or first side chamber and second side chamber; or (b) both the first and second side chambers; determining one or more partition parameters regarding absorption of the test agent into the first side chamber and/or second side chamber; and providing a report having the one or more partition parameters.

In some embodiments, a method can include obtaining data of the one or more partition parameters for at least one test agent; modeling the data with a machine learning system; and obtaining a machine learning model of the subcutaneous absorption model. In some aspects, the method can include modeling absorption of the test agent with the machine learning model of the subcutaneous absorption model.

In some embodiments, the methods can include: obtaining in vitro subcutaneous data with one or more partition parameters of one or more test agents; mapping the one or more partition parameters regarding absorption of the test agent with the in vitro subcutaneous data; and obtaining a correlation model for the subcutaneous absorption model and in vivo data.

In some embodiments, the methods can include screening at least one test agent for being a candidate of subcutaneous administration with the subcutaneous absorption model and analysis of one or more partition parameters.

In some embodiments, a computer-implemented method can include: obtaining partition data of a test agent administered to a ESCAR model of one of the in vitro subcutaneous model embodiments, wherein the partition data includes a measured amount of the test agent in at least one of the: (a) center chamber and/or first side chamber and second side chamber; or (b) both the first and second side chambers, wherein the ESCAR model emulates subcutaneous absorption and release: creating input vectors based on the partition data of the test agent; inputting the input vectors into a machine learning platform; generating one or more predicted partition parameters regarding absorption of the test agent from the center chamber into the first side chamber and/or second side chamber by the machine learning platform, wherein the one or more predicted partition parameters are specific to test agent in the model; and preparing a report that includes the one or more predicted partition parameters.

In some embodiments, the methods can include: training the machine learning platform with partition data of one or more test agents administered one or more times to the center chamber that partition into the first side chamber and/or second side chamber; and providing the trained machine learning platform. In some aspects, the machine learning platform includes a digital model configured to simulate partition parameters in the ESCAR model. In some aspects, the digital model is configured for predicting in vivo subcutaneous injection variables and activities of the test agent. In some aspects, the digital model is configured to predict in vivo absorption pharmacokinetic properties of the test agent. In some aspects, the digital model is configured to simulate test agent aggregation, degradation, and test agent-matrix interactions. In some aspects, the partition data can be based on input factors including matrix concentration, test agent injection volume, test agent injection position, and combinations thereof.

In some embodiments, the machine learning platform models a relationship between the input factors and output responses based on the ESCAR model. In some aspects, the output responses include release percentage of the test agent at a plurality of time points after injection of the test agent into the matrix of the center chamber. In some aspects, the machine learning platform includes machine learning methods with at least one regression model, including support vector machine (SVM), random forest (RF), gradient boosting, and/or multilayer perceptron (MLP). In some aspects, the machine learning method performs a Monte Carlo simulation of release of the test agent from the center chamber.

In some embodiments, the digital model includes a representation of the center chamber being a three-dimensional cuboid composed of a plurality of periodic lattices. In some aspects, each lattice is assigned a coordinate of chamber width, chamber length, and chamber height.

In some embodiments, the present invention can be configured to simulate: a plurality of lattices adjacent to one of the membranes between the center chamber and the first side chamber or second side chamber is configured as a leak lattice; a first plurality of interface lattices between the center chamber and first side chamber are set as leak lattices; a second plurality of interface latices between the center chamber and second side chamber are set as leak lattices, wherein the first plurality is greater than the second plurality; remaining interface lattices of the first plurality and second plurality are set as reflecting lattices; and a plurality of internal lattices (not adjacent to a surface) are set as particle migration lattices.

In some embodiments, the injection into the central chamber is simulated by inserting particles into one or more lattices.

In some embodiments, the Monte Carlo simulation is configured so that time is an arbitrary unit, each particle is chosen at random to determine a probability (q) to stay in current lattice or probability (1−q) to move to an adjacent lattice.

In some embodiments, one or more iterations consider the following: if a chosen lattice is a leak lattice, a specific particle can enter and then leave the lattice; if the chosen lattice is a reflecting lattice, a specific particle can stay at its current lattice; if the chosen lattice is a particle-migration lattice but was already occupied by another particle, the specific particle would stay at its current lattice; or if the chosen lattice is a particle-migration lattice and empty, the specific particle can move to this chosen lattice.

In some embodiments, the ESCAR model includes: the first side chamber being configured as a blood absorption chamber and the second side chamber is configured as a lymph absorption chamber, wherein the second side of the center chamber includes the two spaced apart second openings that have a combined open area less than an open area of the one first opening; or the first side chamber being configured as a first blood absorption chamber and the second side chamber is configured as a second blood absorption chamber, wherein the second side of the center chamber includes the one second opening that has an open area this about the same as an open area of the one first opening.

In some embodiments, one or more non-transitory computer readable media are provided for storing instructions that in response to being executed by one or more processors, cause a computer system to perform operations, the operations comprising the computer-implemented method of one of the computing method embodiments described herein.

In some embodiments, a computer system can include: one or more processors; and one or more non-transitory computer readable media storing instructions that in response to being executed by the one or more processors, cause the computer system to perform operations, the operations comprising the computer-implemented method of one of the computing method embodiments.

In some embodiments, a computer-implemented method can include: obtaining partition data of a test agent administered to a ESCAR model of one of the model claims, wherein the partition data includes a measured amount of the test agent in at least one of the: (a) center chamber and/or first side chamber and second side chamber; or (b) both the first and second side chambers, wherein the ESCAR model emulates subcutaneous absorption and release; modeling the partition data with a digital model of the ESCAR model; generating one or more predicted partition parameters regarding absorption of the test agent from the center chamber into the first side chamber and/or second side chamber; and preparing a report that includes the one or more predicted partition parameters.

In some embodiments, the computer-implemented method can include any steps from one of the embodiments that can be performed on a computer. That is, if the method step is described and can be implemented on a computer, the method step may be part of the computer-implemented method.

In some embodiments, one or more non-transitory computer readable media storing instructions that in response to being executed by one or more processors, cause a computer system to perform operations, the operations comprising the computer-implemented method of one of the computer method embodiments described herein.

In some embodiments, a computer system can include: one or more processors; and one or more non-transitory computer readable media storing instructions that in response to being executed by the one or more processors, cause the computer system to perform operations, the operations comprising the computer-implemented method of one of the methods with method steps that can be implemented on a computer system.

In some embodiments, a system can include the in vitro subcutaneous model device of one of the embodiments; and the computer system of one of the embodiments.

### Examples

ABSplus white and SR-30 were purchased from Stratasys (Edina, MN, USA). Acetaminophen, griseofulvin, and Tween80 were purchased from Sigma-Aldrich (St. Louis, MO, USA). Lecithin (90%, soybean), and SpectraPor® dialysis membranes (MWCO: 50 kDa and 300 kDa) were purchased from Fisher Scientific (Ward Hill, MA, USA). Hyaluronic acid (average molecular weight: 1.64M Da) was purchased from Lifecore Biomedical, Inc. (Chaska, MN, USA). All solvents used in this study were of HPLC analytical grade.

### ESCAR Model Fabrication

The ESCAR layout was drawn using AutoCAD (Autodesk Inc., San Rafael, CA, USA). Each component was printed by a Mojo 3D printer (Stratasys, Inc., Edina, MN, USA) via the fused deposition modeling technology. ABSplus white and SR-30 were utilized as the printing material and the support material, respectively. After printing, the support material was removed by the Ecoworks®-based solution with the aid of a WaveWash 55 Clean system (Stratasys, Inc., Edina, MN, USA). Acetone was sprayed onto the outer and inner surfaces to make the components watertight. Sequentially, the acetone-treated components were placed under (1) ambient temperature overnight; and then (2) 50° C. in a convection oven for at least 72 hr, to remove the acetone residues. Further, all the contact surfaces were smoothened by a series of sandpapers with medium grits and superfine grits.

### Drug Quantification Using HPLC

A Shimadzu HPLC system (Shimadzu Corporation, Kyoto, Japan) equipped with an XBridge™ C18 column (3.5 μM, 4.6×150 mm) was used to quantify the acetaminophen and griseofulvin samples.

For acetaminophen samples, 20 μL was injected and detected at 275 nm. The mobile phase containing 69% of water, 3% of acetic acid, and 28% of methanol (v/v), was kept at a constant flow rate of 0.8 mL/min. The column chamber temperature was set at 27° C. and the detector chamber temperature was maintained at 40° C. For griseofulvin samples, 20 μL was injected and detected at 291 nm. The mobile phase containing 35% of water with 0.1% of trifluoroacetic acid and 65% of acetonitrile (v/v) was kept at a constant flow rate of 1 mL/min. The temperature of both the column chamber and the detector chamber was kept at 40° C.

### ESCAR Drug Binding/Adsorption

ESCAR drug binding/adsorption study was carried out using a procedure as follows: 75 ml of acetaminophen or griseofulvin solution with a known concentration was placed in a ESCAR's acceptor chamber, and the chamber was stored at ambient temperature for 24 hr before the sample collection. The measurements were carried out in triplicate. The percentage of drug recovery was calculated using Eq. 1.

$$Recovery ( \% ) = \frac{Concentration ( after 24 hr in chamber )}{Concentration ( initial )} \times 100 \% \qquad ( Eq . 1 )$$

### Drug Release Tests for Acetaminophen

Acetaminophen solution (10 mg/mL) and the subcutaneous chamber (Version 1; FIG. 2C) were used across different runs. Both the “lymphatic circulation” and “blood circulation” chambers were filled with 75 mL of PBS (pH 7.4), and the subcutaneous chamber was filled with 2.8 mL of HA/PBS solution. At the surrounding areas of the interfaces, a layer of mounting tape was assembled to prevent liquid leakage. Sequentially, the “subcutaneous”/“blood circulation” interface was assembled with a SpectraPor® dialysis membrane (MWCO: 50 kDa), and the “subcutaneous”/“lymphatic circulation” interface was assembled with a SpectraPor® dialysis membrane (MWCO: 300 kDa). All membranes were pre-soaked in DI water for at least 1 h before use. The drug release tests were conducted at 34° C. in a convective oven, with mild magnetic stirring in the “blood circulation” and “lymphatic circulation” chambers. The preset volume of drug solution was manually injected into the “subcutaneous” chamber from the injection port(s) using a 3-mL syringe connected with a 23 G×¾ needle (BD, Franklin Lakes, NJ, USA). A 18-run full factorial experimental design was employed to evaluate three factors, HA concentration (three levels: 2.5, 5, 10 mg/mL), injection volume (three levels: 0.25, 0.5, 1 mL), and injection position/needle tip position to the membrane at the “subcutaneous”/“blood circulation” interface (two levels: 0.2, 0.5 cm). At the pre-set time points, 1.5 mL of aliquots were withdrawn from the “blood circulation” chamber with the replacement of the same volume of PBS. Each run was conducted in triplicate.

### Release Profile Modeling Using Statistical and Machine Learning Methods

A series of statistical and machine learning methods were adopted to model the relationship between the input factors and the output response(s) based on the data generated from the 18-run DoE study. HA concentration (X₁), injection volume (X₂), and injection position to the membrane (X₃) were three input factors. Release percentages (Y) at 2-hr, 4-hr, 6-hr, and 8-hr were selected as the output response(s).

For statistical methods, the data were fit by polynomial equations with the aid of JMP (SAS Institute, Cary, NC, USA). After removing some statistically insignificant second-order and interaction terms, the final format of the polynomial equations was expressed as Eq. 2.

$$Y = intercept + {aX}_{1}^{2} + {bX}_{1} + {cX}_{2} + {dX}_{3} \qquad ( Eq . 2 )$$

A total of four machine learning methods, including support vector machine (SVM), random forest (RF), gradient boosting, and multilayer perceptron (MLP), were also used to develop regression models. The codes were programmed based on the Scikit-Learn module under the Python environment, and hyper-parameters were tuned using the cross-validated grid search method.⁽⁴⁸⁾

### Monte Carlo Simulation

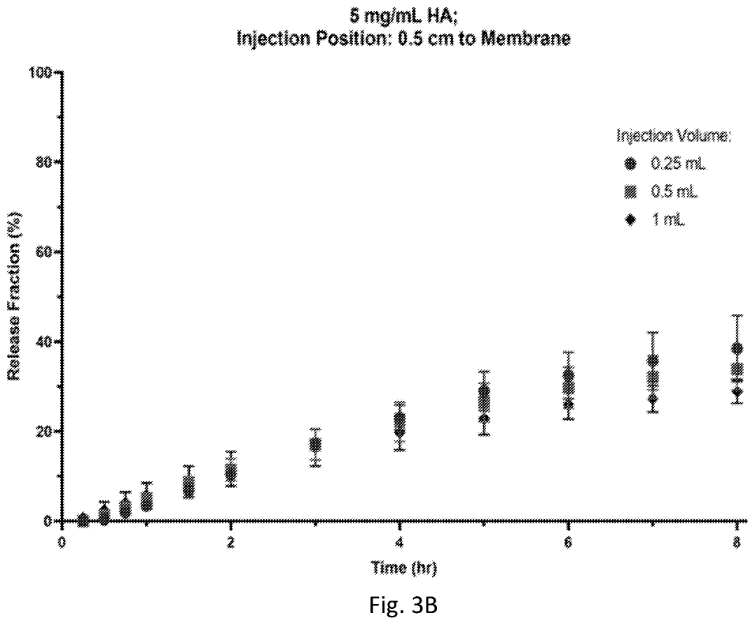

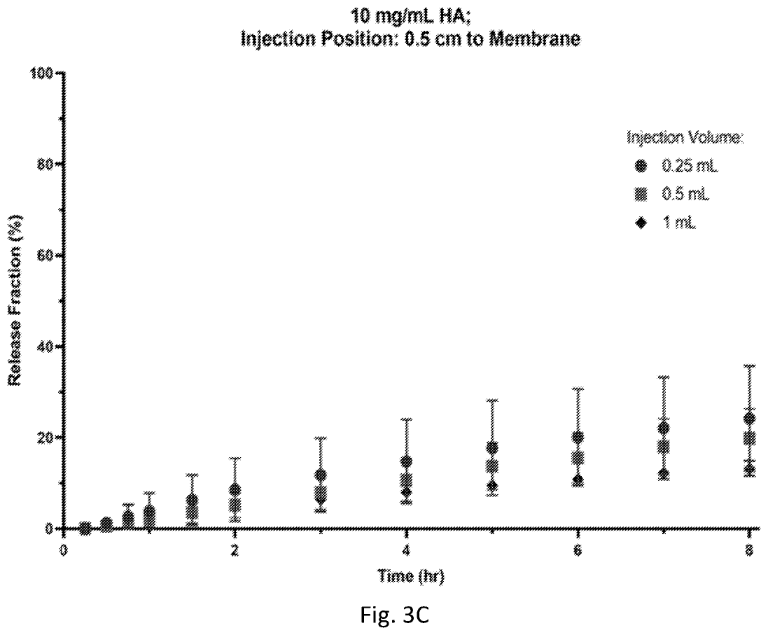

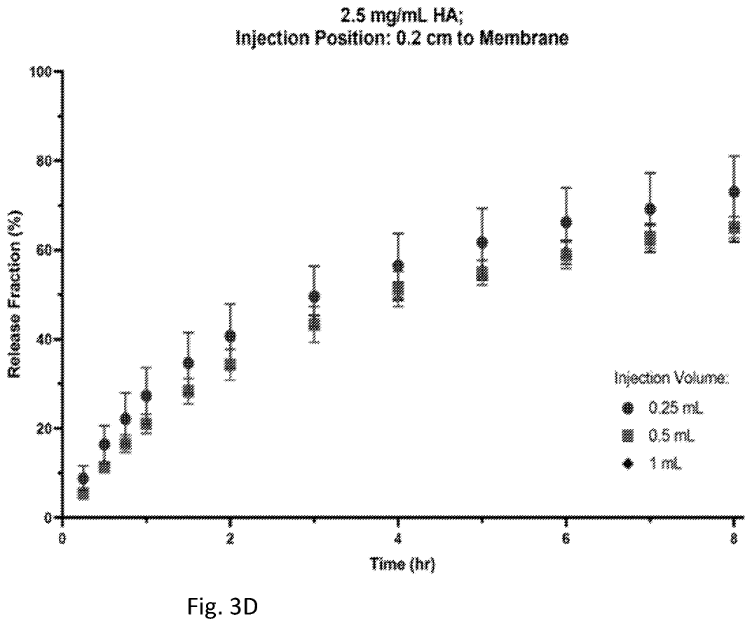

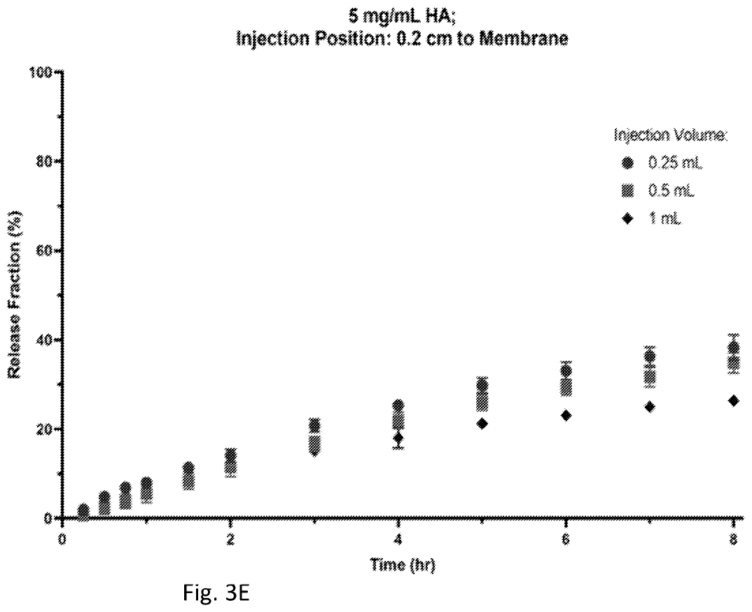

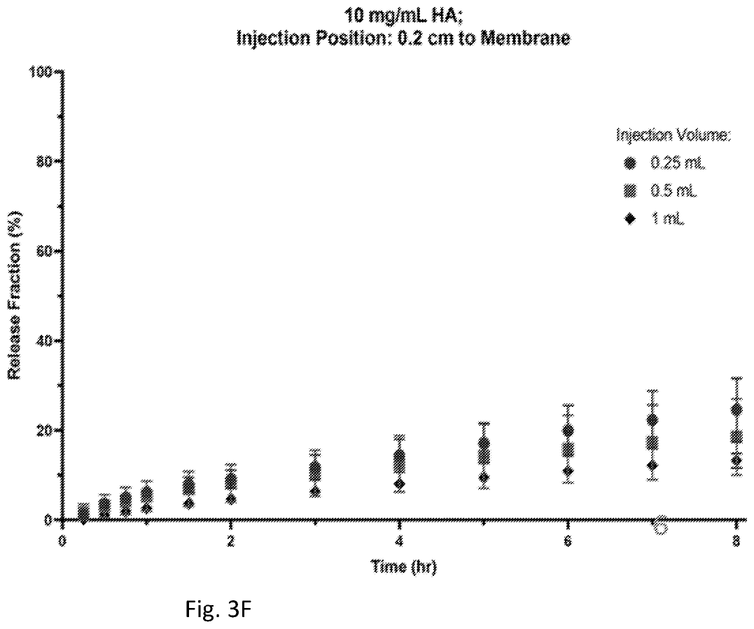

The Monte Carlo method was developed to simulate drug release from the “subcutaneous” chamber. Based on the results of the ESCAR factor screening study, it was rational to speculate that, in high HA solutions (e.g., 5 and 10 mg/mL), drug release was mainly affected by drug migration in the HA solution rather than drug permeation through the membrane, and drug migration in the HA solution could be simulated by the Monte Carlo method. The codes were programmed using Matlab R2018a (MathWorks, Natick, MA, USA). Monte Carlo simulation was employed to further understand the experimental drug release data. Based on the data presented in FIGS. 3A-3F, it was reasonable to speculate that for the 5- and 10-mg/mL HA solution, drug release was mainly controlled by drug migration from the injection site to the membrane rather than drug permeation through the membrane. Also, molecule migration, from the Monte Carlo simulation perspective, could be considered as a particle moving from its original lattice to one of its nearest-neighbor lattices at random.

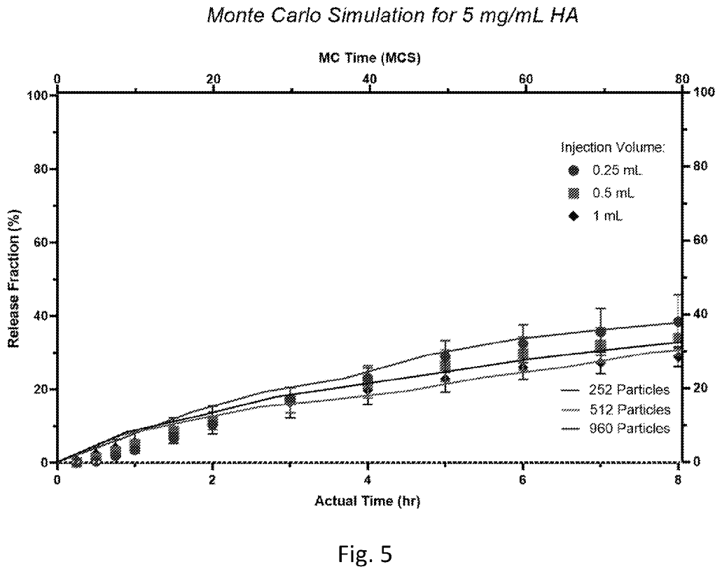

With the insertion of 256, 512, or 960 particles, many central lattices of the cuboid were occupied. By making an analogy between placing particles into lattices and injecting drug solution into the subcutaneous chamber, the simulation result could perhaps explain the experimental finding that why injection position to the membrane was not a critical factor. The reason might be, regardless of the distance of the injection position (the needle tip position) was 0.2 cm or 0.5 cm to the membrane at the “subcutaneous”/“blood circulation” interface, the same central space of the subcutaneous chamber would be occupied after the solution injection. On the contrary, the injection position (the needle tip position) would likely become more impactful if the injection volume was small and could not occupy the majority of the central chamber space. In this scenario, the position of the space generated by the injected solution was directly associated with the injection position (the needle tip position). While particles migrated and eventually left the subcutaneous chamber as a function of MCS, a release profile could be plotted. It was worthwhile to point out that, due to the limited computational power, the system for the Monte Carlo simulation had to be much smaller than the real system. For instance, compared drug molecules in experiments to particles for simulation, for 1 mL of 10 mg/mL acetaminophen solution, there were approximately 1.19E23 acetaminophen molecules, whereas the largest particle number for simulation was 960. Nevertheless, as seen in FIG. 5, using the smaller and simpler systems, the correlations between the simulation data and the actual data were successfully developed, with a time scaling factor of 10 MCS/hr. It was also observed that, for (1) the 5-mg/mL HA medium, the corresponding probability value (q) was 0.45; and (2) for the 10-mg/mL HA medium, the corresponding probability value (q) was 0.8, indicating particle migrated at a slower rate in the 10-mg/mL HA medium. Further, for different injection volume/particle number (e.g., 256, 512, and 960 particles), similar release profiles were obtained, supporting the experimental findings that injection volume was not a critical factor.

### Matrix to Represent the Subcutaneous Chamber Geometry

The subcutaneous chamber could be represented by a three-dimensional cuboid composed of many periodic lattices. Each lattice was assigned by a coordinate (X, Y, Z) and stored in a matrix C. In this study, X, Y, and Z were assigned to chamber width, chamber length, and chamber height, and ranged from 1 to 10, 1 to 32, and 1 to 12 (e.g., arbitrary units, or mm, cm, etc.). To emulate the departure of drug molecules from the subcutaneous chamber through the membranes, the lattices next to the membranes were set as leak lattices. On the “subcutaneous”/“blood circulation” interface, 300 lattices with the coordinates C(10, 2 to 31, 2 to 11) were assigned as leak lattices. Whereas, on the “subcutaneous”/“lymphatic circulation” interface, 20 lattices with the coordinates C(1,2,2 to 11) and C(1,31,2 to 11) were assigned as leak lattices.

The remaining lattices at the boundary surfaces were set as reflecting lattices. Also, the lattices that were not adjacent to the surfaces and were available for particle migration were set as particle-migration lattices. Overall, there were a total of 2720 leak and particle-migration lattices, analogous to the subcutaneous chamber physical volume ˜2.8 mL.

Particle Insertion into the Subcutaneous Chamber

Injecting solution into the subcutaneous chamber was simulated by inserting particles into lattices. After the injection, some lattices at the central region of the chamber were occupied with particles, subjected to the rule that there would be no double occupancy for each lattice. For example, 0.25 mL was simulated by occupying 256 lattices with the coordinates C(3 to 9, 14 to 19, 4 to 9), 0.5 mL was simulated by occupying 512 lattices with the coordinates C(2 to 9, 13 to 20, 3 to 10), and 1 mL was simulated by occupying 960 lattices with the coordinates C(2 to 9, 10 to 21, 2 to 11).

### Particle Migration Inside the Subcutaneous Chamber

For the Monte Carlo simulation, time was recorded by an arbitrary time unit, Monte Carlo Step (MCS). Per each MCS, a particle would be chosen at random, and then this chosen particle would either stay at its current lattice with a probability (q) or attempt to move to one of its nearest-neighbor lattices with a probability (1−q). Hence, a smaller q meant a faster migration rate. Once the particle attempted to move from its current lattice, e.g., the current lattice with the coordinate C(X, Y, Z), to a new lattice, one of the six nearest-neighbor lattices with the coordinates C(X+1, Y, Z), C(X−1, Y, Z), C(X, Y+1, Z), C(X, Y−1, Z), C(X, Y, Z+1), and C(X, Y, Z−1), would be randomly chosen as the potential destination. With this attempt, there were four possible cases: (1) if the chosen lattice was a leak lattice, this particle would enter and then leave the system; (2) if the chosen lattice was a reflecting lattice, this particle would stay at its current lattice (or considered as a step that particle first moved to this lattice, and immediately bounced back to its original lattice); (3) if the chosen lattice was a particle-migration lattice but was already occupied by another particle, this particle would stay at its current lattice; (4) if the chosen lattice was a particle-migration lattice and empty, this particle would move to this new lattice. After this attempt, time was increased by 1/N, where N was the number of particles in the system. The above-mentioned steps would be iterated until all particles departed the chamber. The drug release from the “subcutaneous”/“blood circulation” interface could be plotted by counting the number of particles that were removed from the leak lattices with the coordinates C(10, 2 to 31, 2 to 11).

### In-Vitro-In-Vivo Correlation (IVIVC) Development for Griseofulvin Suspension

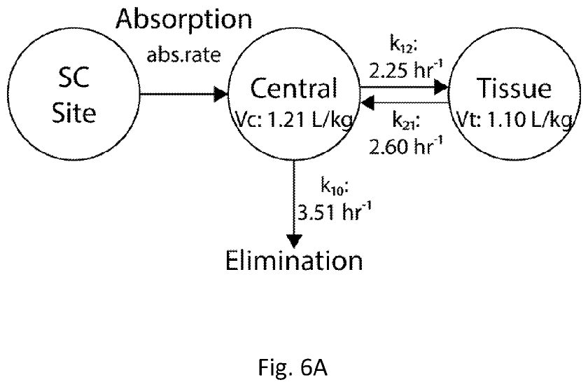

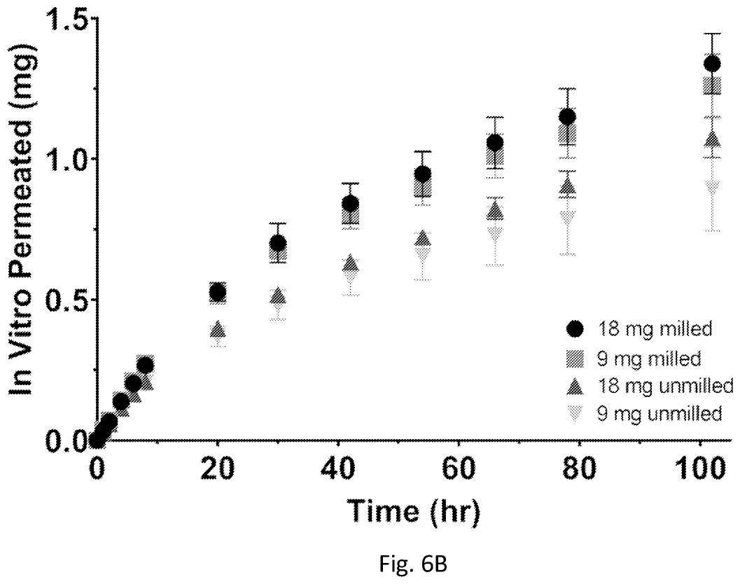

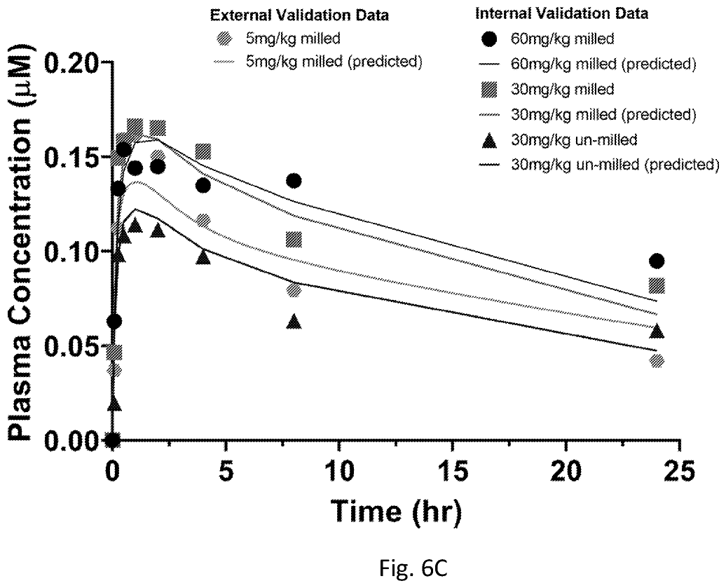

The data of the average rat plasma concentration from 0 to 24 hr were extracted from FIGS. 6B-6C in Chiang et al.'s paper using Origin 2018 (OriginLab Corporation, Northampton, MA, USA).⁽⁴⁹⁾ See FIG. 6A showing the two-compartment PK model for the subcutaneous administration. Referring to Chiang et al.'s paper, the same vendor of the bulk griseofulvin powder and similar preparation methods were used in the present study.⁽⁴⁹⁾ Briefly, to prepare the un-milled suspension, the pre-determined amount of bulk powder was dispensed in 0.5% tween80 (w/w) PBS solution, followed by 5-min sonication. The unmilled suspension was characterized by a Mastersizer 3000 particle size analyzer (Malvern Panalytical, Westborough, MA, USA). To prepare the milled suspension, bulk griseofulvin powder and 0.5-mm zirconium beads were added into a scintillation vial, followed by the addition of 0.5% tween80 (w/w) PBS solution to obtain a final concentration of 50 mg/mL. The suspension was wet-milled under magnetic stirring at 1200 rpm with occasional shaking for 24 hr. The particle size of the milled suspension was characterized by a Zetasizer (Malvern Panalytical, Westborough, MA, USA).

### In Vitro ESCAR Drug Release Tests for Griseofulvin

The subcutaneous chamber (Version 2) was used for the in vitro release tests of griseofulvin un-milled and milled suspensions. For this chamber design, both the front and back interfaces were in contact with the “blood circulation” chambers filled with 75 mL of PBS (pH 7.4). The subcutaneous chamber was filled with 7.5 mL of the O/W emulsion composed of 1.64% (w/v) lecithin and 1 mg/mL HA in PBS solution. Notably, griseofulvin was lipophilic and had a higher partition into the oil phase compared to the aqueous phase. Therefore, to consider the effect of molecule lipophilicity, lecithin was added into the simulated subcutaneous medium to represent the potential drug depots such as adipose tissue and skin lipid. The formulation, dose, and injection volume of our in vitro studies were equivalent to those used in the in vivo studies in Chiang et al.'s paper⁽⁴⁹⁾ In their in vivo study, the rat body weight ranged from 300 to 350 g. To keep the consistency of the dose for our in vitro study, 300 g was selected for in-vitro-in-vivo conversion. For example, if the in vivo dose was 30 mg/kg, the dose of the in vitro release tests was 9 mg. The suspension was injected from the injection port at the center by a 3-mL syringe connected with a 23 G×¾ needle (BD, Franklin Lakes, NJ, USA), and the release test was undertaken at 34° C. with mild magnetic stirring inside the “blood circulation” chambers. For the sampling points before 8-hr, 1.5 mL of aliquots (0.75 mL from each “blood circulation” chamber) were withdrawn with the replacement of PBS; and for the sampling points at 8-hr and beyond, to maintain the concentration gradient between the subcutaneous chamber and the “blood circulation” chambers, 100 mL of aliquots (50 mL from each “blood circulation” chamber) were withdrawn with the replacement of PBS. Each trial was undertaken in triplicate. The release profiles were fit by a three-parameter Weibull equation, expressed as Eq. 3.

$$Y = a \times ( 1 - exp ( - ( \frac{{t}^{b}}{c} ) ) ) \qquad ( Eq . 3 )$$

Where Y was release fraction (%), t was time, and a, b, and c were three constants that could be obtained by curve fitting.

### One-Step Level-A IVIVC Model

Griseofulvin's rat PK profiles could be fit by a two-compartment model.⁽⁴⁹⁾ The mathematical equations corresponding to the change of drug amounts in the central and peripheral compartments (shown in FIG. 6(b)) were described by Eqs. 4 to 6.

$$\frac{dAmt 1}{dt} = Abs . Rate - {k}_{10} Amt 1 - {k}_{12} Amt 1 + {k}_{21} Amt 2 \qquad ( Eq . 4 )$$

$$\frac{dAmt 2}{d t} = {k}_{12} Amt 1 - {k}_{21} Amt 2 \qquad ( Eq . 5 )$$

$$Conc 1 = \frac{Amt 1}{{V}_{c}} \qquad ( Eq . 6 )$$

Where Amt1 and Amt2 were the drug amounts in the central and peripheral compartments at time t, Conc1 was the plasma concentration at time t, V_{c} was the volume of distribution in the central compartment, k₁₀, k₁₂, and k₂₁ were the rate constants for elimination, transfer from the central to the peripheral, and transfer from the peripheral to the central, respectively. The values of V_{c}, k₁₀, k₁₂, and k₂₁ were calculated and reported by Chiang et al.⁽⁴⁹⁾ Abs. Rate, the in vivo absorption rate from the subcutaneous site at time t, was defined and hypothesized to be correlated to the in vitro release rate by a quadratic equation, as expressed by Eq. 7.

$$Abs . Rate = \frac{Dose \times F ( t + Δ t {\vert}_{in vivo} ) - Dose \times F ( t {\vert}_{in vivo} )}{Δ t {\vert}_{in vivo}} = \frac{\begin{matrix}Dose \times F ( {b}_{0} + {b}_{1} \times ( t + Δ t ) + {b}_{2} \times {( t + Δ t )}^{2} {\vert}_{in vitro} ) - \\ Dose \times F ( {b}_{0} + {b}_{1} \times t + {b}_{2} \times {t}^{2} {\vert}_{in vitro} )\end{matrix}}{\begin{matrix}{b}_{0} + {b}_{1} \times ( t + Δ t ) + {b}_{2} \times {( t + Δ t )}^{2} {\vert}_{in vitro} - \\ {b}_{0} + {b}_{1} \times t + {b}_{2} \times {t}^{2} {\vert}_{in vitro}\end{matrix}} \qquad ( Eq . 7 )$$

Where F was the percentage of drug absorbed (in vivo) or the percentage of drug released (in vitro) at time t, and b₀, b₁, and b₂ were time-scaling factors for IVIVC. By tuning b₀, b₁, and b₂, the model that had the best fit of the plasma concentration could be obtained by numerically solving Eqs. 4 to 6, using the custom codes programmed in Matlab R2018a (MathWorks, Natick, CA, USA).

### Parameter Sensitivity Analysis (PSA)

Parameter sensitivity analysis (PSA) was conducted to investigate the impact of each input (PK parameter: k₁₀, k₁₂, k₂₁, and V_{c}) on model output(s). Via varying 10% or 20% for one input parameter, the local parameter sensitivity was estimated by a relative sensitivity coefficient (Rᵢⱼ), as expressed in Eq. 8.⁽²¹⁾

$${R}_{i j} = \frac{\partial {O}_{j}}{\partial {l}_{i}} \times \frac{{I}_{i}}{{O}_{j}} \qquad ( Eq . 8 )$$

where Iᵢ was the local, baseline value of the input parameter i, Oⱼ was the baseline value of the output j, and the term

$$\frac{\partial {O}_{j}}{\partial {I}_{i}}$$

represented the rate of change Oⱼ with respect to Iᵢ. In this study, the sensitivity of Iᵢ would be considered as (a) high if the absolute value |Rᵢⱼ| was ≥0.5; (b) medium if the absolute value |Rᵢⱼ| was ≥0.2 but <0.5; (c) medium if the absolute value |Rᵢⱼ| was ≥0.1 but <0.2; (d) negligible if the absolute value |Rᵢⱼ| was <0.1.⁽⁵⁰⁾

### Drug Binding/Adsorption to ESCAR

Drug binding and/or adsorption to ESCAR was assessed for two small molecule drugs: acetaminophen and griseofulvin. After 24-hr, the recovery (%) of (1) acetaminophen was 99.6±0.2%, and (2) griseofulvin was 95.5±1.7%. Hence, both drugs had no significant drug binding/adsorption to ESCAR. Further, acetaminophen had a slightly higher recovery (%), which might be because acetaminophen was more hydrophilic compared to griseofulvin.

### ESCAR Factor Screening Study Using Acetaminophen Solution

The impact of three factors (HA concentration, injection volume, and injection position to the membrane at the “subcutaneous”/“blood circulation” interface) on drug release was studied via an 18-run full factorial study. However, these studies provide evidence that the model is sufficient for modeling subcutaneous release and absorption. As shown in FIGS. 3A-3F, the increase of HA concentration slowed the drug release, e.g., for the center position injection, the release fraction (%) at 8-hr was ˜80% in PBS, ˜60% in 2.5 mg/mL HA solution, ˜30% in 5 mg/mL HA solution, ˜15% in 10 mg/mL HA solution. From the molecular level perspective, drug release from the subcutaneous chamber consisted of two steps: (a) drug molecules migrated from the position after the injection to the absorption site (membrane), and then (b) molecules permeated through the membrane to the acceptor chamber. The release data indicated that if the subcutaneous medium was PBS or low HA solutions (e.g., 2.5 mg/mL), drug release was mainly controlled by drug permeation through the membrane; whereas, for high HA solutions (e.g., 5 mg/mL and 10 mg/mL), drug release was predominantly controlled by drug migration from the injection site to the absorption site.

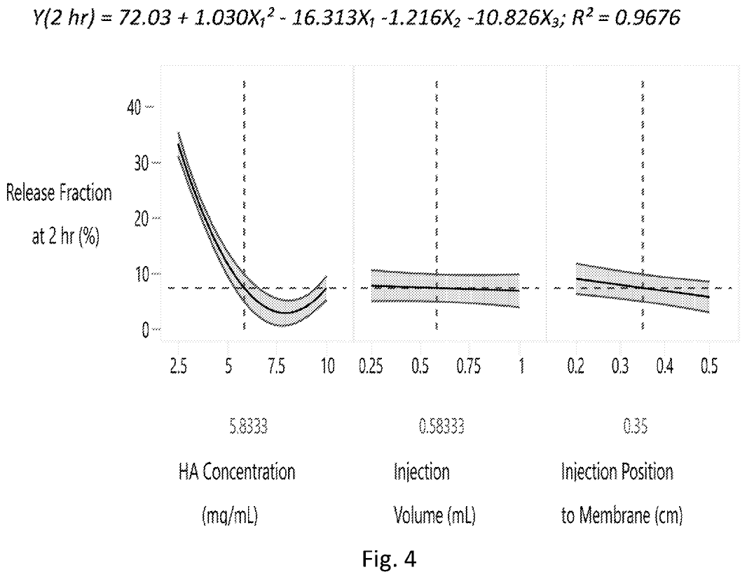

A series of statistical and machine learning methods were developed to model the relationship between the input factors and the output response (drug release). The DoE study data was used for model training and validation. As seen in FIG. 4, the release fraction (%) at 2-hours is shown (but could be other time points such as 4-, 6-, and 8-hr) versus three factors could be fit by polynomial equations (in the format of Eq. 2) with acceptable fitness. The prediction profiler plots in FIG. 4 suggested that release fraction underwent a rapid decline as HA concentration increased from 2.5 mg/mL to 6 mg/mL, and then gradually leveled out while HA concentration raised above 6 mg/mL. Conversely, the release fraction was not substantially changed by injection volume and injection position to the membrane.

Unlike the statistical models that require manually-set rules and instructions, e.g., assigning a 2^{nd}-order polynomial to depict the curvature of the response surface, machine learning methods could learn the data on their own and develop a model(s) with better predictability, although simultaneously the model interpretability might be compromised. As seen in Table 1, all machine learning models (SVM, RF, gradient boosting, and MLP) had higher R²-score values than polynomial equations, suggesting that the training data was fit better by the machine learning models. To avoid the potential overfitting issue, a 4-fold cross-validation method was developed, where the three folds of the whole dataset were used for model training, and the remaining one-fold was used for model validation. Based on the high R²-score values gained for both training and validation datasets, it was concluded that all developed machine learning models could learn the training dataset to develop the predictive model(s) and have good generalization power to predict new data.

### IVIVC

It was of value to explore ESCAR's capability in predicting in vivo PK properties and developing IVIVC models. Using the methods listed in Chiang et al.'s paper,⁽⁴⁹⁾ our in-house-made un-milled and milled suspensions had similar particle sizes as theirs (D50 of our un-milled suspension: 26.2±2.06 μm vs. D50 of their un-milled suspension: 21.9 μm; D50 of our milled suspension: 1.5±0.2 μm vs. D50 of their milled suspension: 1.7 μm). To attain a release profile for a suspension in ESCAR, drug molecules needed to first dissolve and then the dissolved molecules migrated to the membrane, followed by the permeation through the membrane to the acceptor chamber. The in vitro drug release amount (mg) versus time was presented in FIG. 6B. As seen, the milled suspension released faster than the un-milled suspension, e.g., given a 9-mg dose, at 102 hr, 1.34 mg (the milled suspension) was 50% higher than 0.89 mg (the unmilled suspension), indicating the advantage of micronization on dissolution rate enhancement. Further, the 1.5-mg dose of the milled suspension provided a faster release than the 9-mg dose of the unmilled suspension. Also, while the dose of the milled suspension increased from 9 mg to 18 mg, the drug release amount enhanced marginally. Another finding was that the in vitro release in ESCAR was slower than the in vivo dissolution/absorption in Chiang et al.'s paper. For instance, according to the data presented in FIG. 5 of Chiang et al.'s paper, for the 9-mg dose of the un-milled suspension, the in vivo drug absorption amount at 24 hr was similar to the in vitro drug release amount at 54 hr.

To build the IVIVC model, three dose/formulation combinations (9-mg/un-milled, 9-mg/milled, and 18-mg/milled) were used as the internal-validation data, and the in vitro release profiles were plotted in FIG. 6B. The high R²-score values, listed in Table 2, suggested that release profile (%) could be fit by the Weibull function (Eq. 3). As presented in FIG. 6C, a LEVEL A IVIVC model was developed. Table 3 listed the values of Cₘₐₓ, AUC₀₋₂₄ₕᵣ, and the prediction error (% PE). As a result, the model passed the internal validation, and the average absolute internal % PE for Cₘₐₓ was 3.6%, and for AUC₀₋₂₄ₕᵣ was 2.9%. In addition, to make the model more conclusive, the 1.5-mg dose of the milled suspension was evaluated as the external validation. The % PE values for Cₘₐₓ and AUC₀₋₂₄ₕᵣ were 14.9% and −11.5%, respectively. Using this IVIVC model, for a new griseofulvin suspension formulation, only in vitro release tests would need to be conducted in ESCAR, and costly and time-consuming in vivo studies might be waived. Therefore, with this strategy, the 3R (reduce/refine/replace) principle for preclinical species was implemented. Promisingly, using the same methodology, it was also possible to develop an IVIVC model based on human PK data and apply it to various studies such as new formulation development and bioequivalence studies.

In some embodiments, a method of modeling subcutaneous absorption is provided. Such a method can include: providing the subcutaneous absorption model of one of the embodiments; introducing a test agent in a first amount into the matrix material in the center chamber; allowing the test agent to partition into the first side chamber and/or the second side chamber; measuring an amount of the test agent in at least one of the: (a) center chamber and/or first side chamber and second side chamber; or (b) both the first and second side chambers; determining one or more partition parameters regarding absorption of the test agent into the first side chamber and/or second side chamber; and providing a report having the one or more partition parameters. In some aspects, data obtained from the model is processed by a computing system having executable instructions for obtaining the one or more partition parameters regarding absorption of the test agent. The data can be processed through a computer simulation as described herein. The data can be computationally modeled with a computer model of the physical subcutaneous absorption model. The data can be processed through a machine learning system as described herein.

In some embodiments, the methods can include: obtaining data of the one or more partition parameters for at least one test agent; modeling the data with a machine learning system; and obtaining a machine learning model of the subcutaneous absorption model.

In some embodiments, the method can include modeling absorption of the test agent with the machine learning model of the subcutaneous absorption model.

In some embodiments, the methods can include: obtaining in vitro subcutaneous data with one or more partition parameters of one or more test agents; mapping the one or more partition parameters regarding absorption of the test agent with the in vitro subcutaneous data; and obtaining a correlation model for the subcutaneous absorption model and in vivo data.

In some embodiments, the methods can include screening at least one test agent for being a candidate of subcutaneous administration with the subcutaneous absorption model and analysis of one or more partition parameters.

### Protein Study

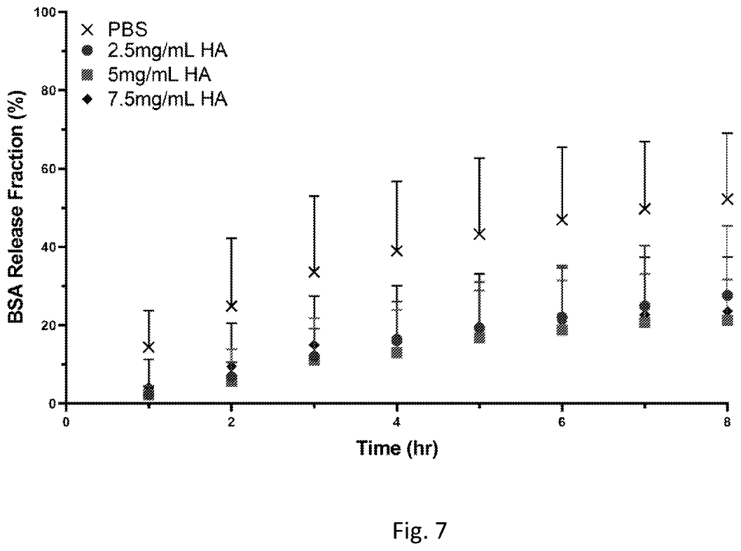

A study of applying the in vitro ESCAR device for protein drug release test was performed. The data for the in vitro ESCAR drug release tests for Bovine serum albumin (BSA) is provided in FIG. 7. The release experiments of BSA were studied using the ESCAR device (FIG. 1A). The open area of the “subcutaneous”/“blood circulation” interface was covered by a 50-kDa MWCO dialysis membrane, and the left and right open areas of the “subcutaneous”/“lymphatic circulation” interface were covered by a customized membrane prepared by puncturing an array of pores on a 300-KDa MWCO dialysis membrane using an SDARA® microneedle derma roller (Sdara Skincare, CA, USA). Each open area contained 30 diamond-shaped punctured pores (average size of 0.011-cm in length and 0.0054-cm in width) and the interpore distance was 0.1 cm horizontally and 0.19 cm vertically. It was speculated that BSA molecules could penetrate these open pores instantaneously, and therefore, the BSA release rate was primarily limited by the migration from the injection site to the membranes. This design may partially reflect the fact of the SC local migration and the subsequent lymphatic uptake. During the device setup, the three chambers were properly aligned, and the membranes pre-soaked in DI water for at least an hour were installed. Further, to prevent the potential leakage, at the surrounding area of the interfaces on the “blood circulation” chamber or the “lymphatic circulation” chamber, a layer of mounting tape was attached, followed by the tightening by clamps.

Consecutively, 2.8 mL of HA/PBS solution was added into the subcutaneous (center) chamber, and the other two chambers were respectively filled with 75 mL of PBS solution. All the injection and liquid-addition ports at the top of the subcutaneous chamber were tightly sealed by at least two layers of waterproof seal tapes to avoid liquid flush-out from these ports under pressure. The whole ESCAR system was placed in a convective oven at 34° C. Both the “blood circulation” chamber and the “lymphatic circulation” chamber were subjected to gentle magnetic stirring.

To begin the drug release test, a predetermined volume of BSA solution (30 mg/mL) was injected into the subcutaneous chamber from the port at the center using a 3-mL syringe connected with a 23 G×¾ needle (BD, NJ, USA). At each sampling point, 1.5 mL of aliquots were taken from the “lymphatic circulation) chamber and the same volume of PBS were added back to the chamber.”

Accordingly, FIG. 7 shows the BSA release profiles after the 0.5-mL injection of BSA solution (30 mg/mL) into the subcutaneous chamber filled with PBS or HA solution. As seen, the BSA release in PBS was significantly faster than that in HA solutions but the BSA release was relatively independent of the change of HA concentration, e.g., the release fraction (%) was ˜52% in PBS, ˜28% in 2.5-mg/mL HA solution, ˜21% in 5-mg/mL HA solution, and ˜24% in 7.5-mg/mL HA solution.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In one embodiment, the present methods can include aspects performed on a computing system. As such, the computing system can include a memory device that has the computer-executable instructions for performing the method. The computer-executable instructions can be part of a computer program product that includes one or more algorithms for performing any of the methods of any of the claims.

In one embodiment, any of the operations, processes, methods, or steps described herein can be implemented as computer-readable instructions stored on a computer-readable medium. The computer-readable instructions can be executed by a processor of a wide range of computing systems from desktop computing systems, portable computing systems, tablet computing systems, hand-held computing systems as well as network elements, base stations, femtocells, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

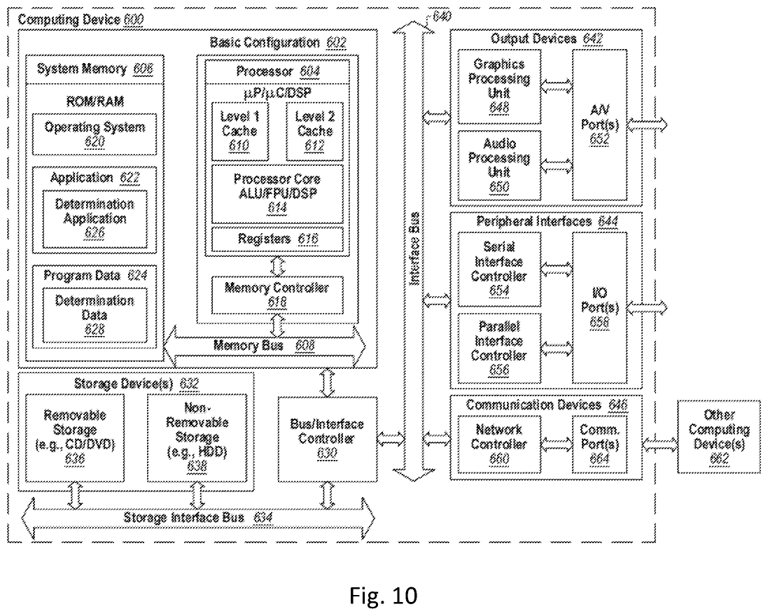

FIG. 10 shows an example computing device 600 that is arranged to perform any of the computing methods described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including but not limited to a microprocessor (μP), a microcontroller (μC), a digital signal processor (DSP), or any combination thereof. Processor 604 may include one more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations memory controller 618 may be an internal part of processor 604.

Depending on the desired configuration, system memory 606 may be of any type including but not limited to volatile memory (such as RAM), non-volatile memory (such as ROM, flash memory, etc.) or any combination thereof. System memory 606 may include an operating system 620, one or more applications 622, and program data 624. Application 622 may include a determination application 626 that is arranged to perform the functions as described herein including those described with respect to methods described herein. Program Data 624 may include determination information 628 that may be useful for analyzing the contamination characteristics provided by the sensor unit 240. In some embodiments, application 622 may be arranged to operate with program data 624 on operating system 620 such that the work performed by untrusted computing nodes can be verified as described herein. This described basic configuration 602 is illustrated in FIG. 6 by those components within the inner dashed line.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the term “module” or “component” can refer to software objects or routines that execute on the computing system. The different components, modules, engines, and services described herein may be implemented as objects or processes that execute on the computing system (e.g., as separate threads). While the system and methods described herein are preferably implemented in software, implementations in hardware or a combination of software and hardware are also possible and contemplated. In this description, a “computing entity” may be any computing system as previously defined herein, or any module or combination of modulates running on a computing system.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### REFERENCES

1. Hernindez-Ruiz V, Forestier E, Gavazzi G, Ferry T, Grégoire N, Breilh D, et al. Subcutaneous Antibiotic Therapy: The Why, How, Which Drugs and When. Journal of the American Medical Directors Association. 2021; 22(1):50-5.e6.
2. Neklesa T K, Winkler J D, Crews C M. Targeted protein degradation by PROTACs. Pharmacology & Therapeutics. 2017; 174:138-44.
3. Kumar A. Insulin degludec/liraglutide: innovation-driven combination for advancement in diabetes therapy. Expert Opinion on Biological Therapy. 2014; 14(6):869-78.
4. Guo Y, Lei K, Tang L. Neoantigen Vaccine Delivery for Personalized Anticancer Immunotherapy. Frontiers in immunology. 2018; 9:1499-.
5. Sahin U, Tureci Ö. Personalized vaccines for cancer immunotherapy. Science. 2018; 359(6382):1355-60.
6. Kinnunen H M, Mrsny R J. Improving the outcomes of biopharmaceutical delivery via the subcutaneous route by understanding the chemical, physical and physiological properties of the subcutaneous injection site. Journal of Controlled Release. 2014; 182:22-32.
7. Viola M, Sequeira J, Seiça R, Veiga F, Serra J, Santos A C, et al. Subcutaneous delivery of monoclonal antibodies: How do we get there? Journal of Controlled Release. 2018; 286:301-14.
8. Zou P, Wang F, Wang J, Lu Y, Tran D, Seo S K. Impact of injection sites on clinical pharmacokinetics of subcutaneously administered peptides and proteins. Journal of Controlled Release. 2021; 336:310-21.
9. Wang W. Advanced protein formulations. Protein Science. 2015; 24(7):1031-9.
10. Marschall C, Witt M, Hauptmeier B, Friess W. Powder suspensions in non-aqueous vehicles for delivery of therapeutic proteins. European Journal of Pharmaceutics and Biopharmaceutics. 2021; 161:37-49.
11. Li J, Mooney D J. Designing hydrogels for controlled drug delivery. Nature Reviews Materials. 2016; 1(12):16071.
12. Cai S, Yang Q, Bagby T R, Forrest M L. Lymphatic drug delivery using engineered liposomes and solid lipid nanoparticles. Advanced drug delivery reviews. 2011; 63(10-11):901-8.
13. Richter W F, Jacobsen B. Subcutaneous Absorption of Biotherapeutics: Knowns and Unknowns. Drug Metabolism and Disposition. 2014; 42(11):1881-9.
14. Collins D S, Sánchez-Félix M, Badkar A V, Mrsny R. Accelerating the development of novel technologies and tools for the subcutaneous delivery of biotherapeutics. Journal of Controlled Release. 2020; 321:475-82.
15. Turner M R, Balu-Iyer S V. Challenges and Opportunities for the Subcutaneous Delivery of Therapeutic Proteins. J Pharm Sci. 2018; 107(5):1247-60.
16. Azarmi S, Roa W, Labenberg R. Current perspectives in dissolution testing of conventional and novel dosage forms. International Journal of Pharmaceutics. 2007; 328(1):12-21.
17. Hens B, Bermejo M, Tsume Y, Gonzalez-Alvarez I, Ruan H, Matsui K, et al. Evaluation and optimized selection of supersaturating drug delivery systems of posaconazole (BCS class 2b) in the gastrointestinal simulator (GIS): An in vitro-in silico-in vivo approach. European Journal of Pharmaceutical Sciences. 2018; 115:258-69.
18. Hens B, Bermejo M, Augustijns P, Cristofoletti R, Amidon G E, Amidon G L. Application of the Gastrointestinal Simulator (GIS) Coupled with In Silico Modeling to Measure the Impact of Coca-Cola® on the Luminal and Systemic Behavior of Loratadine (BCS Class 2b). Pharmaceutics. 2020; 12(6):566.
19. Li D, Chow P Y, Lin T P, Cheow C, Li Z, Wacker M G. Simulate SubQ: The Methods and the Media. Journal of Pharmaceutical Sciences. 2021.
20. Janas C, Mast M-P, Kirsamer L, Angioni C, Gao F, Mantele W, et al. The dispersion releaser technology is an effective method for testing drug release from nanosized drug carriers. European Journal of Pharmaceutics and Biopharmaceutics. 2017; 115:73-83.
21. Gao G F, Ashtikar M, Kojima R, Yoshida T, Kaihara M, Tajiri T, et al. Predicting drug release and degradation kinetics of long-acting microsphere formulations of tacrolimus for subcutaneous injection. Journal of Controlled Release. 2021; 329:372-84.
22. Gao G F, Thurn M, Wendt B, Parnham M J, Wacker M G. A sensitive in vitro performance assay reveals the in vivo drug release mechanisms of long-acting medroxyprogesterone acetate microparticles. International Journal of Pharmaceutics. 2020; 586:119540.
23. Lou H, Berkland C, Hageman M J. Simulating particle movement inside subcutaneous injection site simulator (SCISSOR) using Monte-Carlo method. International Journal of Pharmaceutics. 2021; 605:120824.
24. Kinnunen H M, Sharma V, Contreras-Rojas L R, Yu Y, Alleman C, Sreedhara A, et al. A novel in vitro method to model the fate of subcutaneously administered biopharmaceuticals and associated formulation components. Journal of Controlled Release. 2015; 214:94-102.
25. Bown H K, Bonn C, Yohe S, Yadav D B, Patapoff T W, Daugherty A, et al. In vitro model for predicting bioavailability of subcutaneously injected monoclonal antibodies. Journal of Controlled Release. 2018; 273:13-20.
26. Shan L, Mody N, Sormani P, Rosenthal K L, Damschroder M M, Esfandiary R. Developability Assessment of Engineered Monoclonal Antibody Variants with a Complex Self-Association Behavior Using Complementary Analytical and in Silico Tools. Molecular Pharmaceutics. 2018; 15(12):5697-710.
27. Thati S, McCallum M, Xu Y, Zheng M, Chen Z, Dai J, et al. Novel Applications of an In Vitro Injection Model System to Study Bioperformance: Case Studies with Different Drug Modalities. Journal of Pharmaceutical Innovation. 2020; 15(2):268-80.
28. Kagan L. Pharmacokinetic Modeling of the Subcutaneous Absorption of Therapeutic Proteins. Drug Metabolism and Disposition. 2014; 42(11):1890-905.
29. Braverman I M. The Cutaneous Microcirculation. Journal of Investigative Dermatology Symposium Proceedings. 2000; 5(1):3-9.
30. Frayn K N, Karpe F. Regulation of human subcutaneous adipose tissue blood flow. International Journal of Obesity. 2014; 38(8):1019-26.
31. Redondo PdAG, Gubert F, Zaverucha-do-Valle C, Dutra T P P, Ayres-Silva JdP, Fernandes N, et al. Lymphatic vessels in human adipose tissue. Cell and Tissue Research. 2020; 379(3):511-20.
32. Skobe M, Detmar M. Structure, Function, and Molecular Control of the Skin Lymphatic System. Journal of Investigative Dermatology Symposium Proceedings. 2000; 5(1):14-9.
33. Porter C J H, Edwards G A, Charman S A. Lymphatic transport of proteins after s.c. injection: implications of animal model selection. Advanced Drug Delivery Reviews. 2001; 50(1):157-71.
34. Porter C J H, Charman S A. Lymphatic Transport of Proteins After Subcutaneous Administration. Journal of Pharmaceutical Sciences. 2000; 89(3):297-310.
35. McLennan D N, Porter C J H, Charman S A. Subcutaneous drug delivery and the role of the lymphatics. Drug Discovery Today: Technologies. 2005; 2(1):89-96.
36. Komarova Y, Malik A B. Regulation of Endothelial Permeability via Paracellular and Transcellular Transport Pathways. Annual Review of Physiology. 2010; 72(1):463-93.
37. Charman S A, McLennan D N, Edwards G A, Porter C J H. Lymphatic Absorption Is a Significant Contributor to the Subcutaneous Bioavailability of Insulin in a Sheep Model. Pharm Res. 2001; 18(11):1620-6.
38. Supersaxo A, Hein W R, Steffen H. Effect of Molecular Weight on the Lymphatic Absorption of Water-Soluble Compounds Following Subcutaneous Administration. Pharm Res. 1990; 7(2):167-9.
39. Charman S A, Segrave A M, Edwards G A, Porter C J H. Systemic Availability and Lymphatic Transport of Human Growth Hormone Administered by Subcutaneous Injection. Journal of Pharmaceutical Sciences. 2000; 89(2):168-77.
40. McLennan D N, Porter C J H, Edwards G A, Martin S W, Heatherington A C, Charman S A. Lymphatic Absorption Is the Primary Contributor to the Systemic Availability of Epoetin Alfa following Subcutaneous Administration to Sheep. Journal of Pharmacology and Experimental Therapeutics. 2005; 313(1):345-51.
41. McLennan D N, Porter C J H, Edwards G A, Heatherington A C, Martin S W, Charman S A. The Absorption of Darbepoetin Alfa Occurs Predominantly via the Lymphatics Following Subcutaneous Administration to Sheep. Pharm Res. 2006; 23(9):2060-6.
42. Kota J, Machavaram K K, McLennan D N, Edwards G A, Porter C J H, Charman S A. Lymphatic Absorption of Subcutaneously Administered Proteins: Influence of Different Injection Sites on the Absorption of Darbepoetin Alfa Using a Sheep Model. Drug Metabolism and Disposition. 2007; 35(12):2211-7.
43. Sawdon M, Kirkman E. Capillary dynamics and the interstitial fluid-lymphatic system. Anaesthesia & Intensive Care Medicine. 2017; 18(6):309-15.
44. Haggerty A, Nirmalan M. Capillary dynamics, interstitial fluid and the lymphatic system. Anaesthesia & Intensive Care Medicine. 2019; 20(3):182-9.
45. Collins D S, Kourtis L C, Thyagarajapuram N R, Sirkar R, Kapur S, Harrison M W, et al. Optimizing the Bioavailability of Subcutaneously Administered Biotherapeutics Through Mechanochemical Drivers. Pharm Res. 2017; 34(10):2000-11.
46. McDonald T A, Zepeda M L, Tomlinson M J, Bee W H, Ivens I A. Subcutaneous administration of biotherapeutics: current experience in animal models. Current opinion in molecular therapeutics. 2010; 12(4):461-70.
47. Turner M R, Balu-Iyer S V. Challenges and Opportunities for the Subcutaneous Delivery of Therapeutic Proteins. Journal of Pharmaceutical Sciences. 2018.
48. Pedregosa F, Varoquaux G, Gramfort A, Michel V, Thirion B, Grisel 0, et al. Scikit-learn: Machine Learning in Python. J Mach Learn Res. 2011; 12(null):2825-30.
49. Chiang P-C, Nagapudi K, Fan P W, Liu J. Investigation of Drug Delivery in Rats via Subcutaneous Injection: Case Study of Pharmacokinetic Modeling of Suspension Formulations. Journal of Pharmaceutical Sciences. 2019; 108(1):109-19.
50. Najjar A, Schepky A, Krueger C-T, Dent M, Cable S, Li H, et al. Use of Physiologically-Based Kinetics Modelling to Reliably Predict Internal Concentrations of the UV Filter, Homosalate, After Repeated Oral and Topical Application. Frontiers in Pharmacology. 2022; 12.

TABLE 1
Tables
Summary of the goodness of fit of different regression models for acetaminophen release profiles
Regression Score: R²

| | Y(2 hr) | | Y(4 hr) | | Y(6 hr) | | Y(8 hr) | |
|---|---|---|---|---|---|---|---|---|
| Predictive | CrossValidation? | | CrossValidation? | | CrossValidation? | | CrossValidation? | |
| Models | No | Yes: 4 Fold | No | Yes: 4 Fold | No | Yes: 4 Fold | No | Yes: 4 Fold |
| Polynomial | 0.9676 | NA | 0.9836 | NA | 0.9817 | NA | 0.9789 | NA |
| Equation | | | | | | | | |
| | | Dataset | | Dataset | | Dataset | | Dataset |
| | | Training: | | Training: | | Training: | | Training: |
| SVM | 0.9845 | 0.9944 | 0.9956 | 0.9978 | 0.9963 | 0.9975 | 0.9947 | 0.9959 |
| | | Validation: | | Validation: | | Validation: | | Validation: |
| | | 0.9283 | | 0.9828 | | 0.9797 | | 0.9776 |
| | | Dataset | | Dataset | | Dataset | | Dataset |
| | | Training: | | Training: | | Training: | | Training: |
| RF | 0.9896 | 0.9882 | 0.9950 | 0.9950 | 0.9838 | 0.9933 | 0.9956 | 0.9926 |
| | | Validation: | | Validation: | | Validation: | | Validation: |
| | | 0.9146 | | 0.9695 | | 0.9597 | | 0.9520 |
| | | Dataset | | Dataset | | Dataset | | Dataset |
| | | Training: | | Training: | | Training: | | Training: |
| Gradient | 0.9960 | 0.9970 | 0.9987 | 0.9991 | 0.9999 | 0.99996 | 0.9986 | 0.9989 |
| Boosting | | | | | | | | |
| | | Validation: | | Validation: | | Validation: | | Validation: |
| | | 0.9290 | | 0.9774 | | 0.9660 | | 0.9610 |
| | | Dataset | | Dataset | | Dataset | | Dataset |
| | | Training: | | Training: | | Training: | | Training: |
| MLP | 0.9976 | 0.9973 | 0.9926 | 0.9975 | 0.9995 | 0.9996 | 0.9984 | 0.9974 |

| Validation: | Validation: | Validation: | Validation: |
|---|---|---|---|
| 0.9370 | 0.9758 | 0.9690 | 0.9611 |

TABLE 2
Parameters of Weibull function for fitting in vitro
release profiles of griseofulvin suspensions
$Weibull equation : Y = a \times ( 1 - exp ( - ( \frac{{t}^{b}}{c} ) ) )$

| | | a | b | c | R² |
|---|---|---|---|---|---|
| 18 | mg (60 mg/kg) milled | 10.08 | 0.8335 | 36.61 | 0.9985 |
| 9 | mg (30 mg/kg) milled | 18.95 | 0.8034 | 31.66 | 0.9985 |
| 9 | mg (30 mg/kg) unmilled | 13.90 | 0.7747 | 29.28 | 0.9993 |
| 1.5 | mg (5 mg/kg) milled | 105.5 | 0.7698 | 33.28 | 0.9995 |

TABLE 3
Internal and external validation for the
IVIVC model of griseofulvin suspensions

| | Data | Cmax (μM) | | | AUC0-24 (μM × h) | | |
|---|---|---|---|---|---|---|---|
| Dose/Formulation | Type | Obs. | Pred. | % PE | Obs. | Pred. | % PE |
| 18 mg (60 mg/kg) | Internal | 0.154 | 0.163 | −5.7 | 2.95 | 2.77 | 6.1 |
| milled | | | | | | | |
| 9 mg (30 mg/kg) | Internal | 0.166 | 0.165 | 0.9 | 2.64 | 2.63 | 0.5 |
| milled | | | | | | | |
| 9 mg (30 mg/kg) | Internal | 0.114 | 0.109 | 4.4 | 1.71 | 1.74 | −2.2 |
| unmilled | | | | | | | |
| Average absolute % | internal | | | 3.6 | | | 2.9 |
| PE for validation | | | | | | | |
| 1.5 mg (5 mg/kg) | External | 0.161 | 0.137 | 14.9 | 1.91 | 2.13 | −11.5 |
| milled | | | | | | | |

TABLE 4
Statistical Model vs. Machine Learning Model
Regression Score: R²

| | Y(2 hr) | | Y(8 hr) | |
|---|---|---|---|---|
| Predictive | CrossValidation? | | CrossValidation? | |
| Models | No | Yes: 4 Fold | No | Yes: 4 Fold |
| Polynomial | 0.9676 | NA | 0.9789 | NA |
| Equation | | | | |
| SVM | 0.9845 | Dataset | 0.9947 | Dataset |
| | | Training: | | Training: |
| | | 0.9944 | | 0.9959 |
| | | Test: | | Test: |
| | | 0.9283 | | 0.9776 |
| RF | 0.9896 | Dataset | 0.9956 | Dataset |
| | | Training: | | Training: |
| | | 0.9882 | | 0.9926 |
| | | Test: | | Test: |
| | | 0.9146 | | 0.9520 |
| Gradient | 0.9960 | Dataset | 0.9986 | Dataset |
| Boosting | | Training: | | Training: |
| | | 0.9970 | | 0.9989 |
| | | Test: | | Test: |
| | | 0.9290 | | 0.9610 |
| MLP | 0.9976 | Dataset | 0.9984 | Dataset |
| | | Training: | | Training: |
| | | 0.9973 | | 0.9974 |
| | | Test: | | Test: |
| | | 0.9370 | | 0.9611 |

TABLE 5
internal and external validation for the IVIC model,
referring to FIGS. 6A-6C
Internal and External Validation for the IVIVC Model

| | Data | Cmax (μM) | | | AUC0-24 (μM × h) | | |
|---|---|---|---|---|---|---|---|
| Dose/Formulation | Type | Obs. | Pred. | % PE | Obs. | Pred. | % PE |
| 18 mg (60 mg/kg) | Internal | 0.154 | 0.163 | −5.7 | 2.95 | 2.77 | 6.1 |
| milled | | | | | | | |
| 9 mg (30 mg/kg) | Internal | 0.166 | 0.165 | 0.9 | 2.64 | 2.63 | 0.5 |
| milled | | | | | | | |
| 9 mg (30 mg/kg) | Internal | 0.114 | 0.109 | 4.4 | 1.71 | 1.74 | −2.2 |
| unmilled | | | | | | | |
| Average absolute % | internal | | | 3.6 | | | 2.9 |
| PE for validation | | | | | | | |
| 1.5 mg (5 mg/kg) | External | 0.161 | 0.138 | 14.4 | 1.91 | 2.16 | −12.9 |
| milled | | | | | | | |

1. A modular in vitro device configured as a subcutaneous absorption model, comprising:
a center chamber formed by a center chamber body having a first side with at least one first opening and a second side with at least one second opening;
a matrix material configured to be included in the center chamber during measurement of absorption of a test agent;
a first side chamber formed by a first side chamber body having a first open side that is configured to couple with the first side of the center chamber, the first open side having at least one first side opening configured to fluidly couple with the center chamber through the at least one first opening when the first side chamber is mounted to the center chamber;
at least one first membrane configured to be positioned between the first side of the center chamber and first open side of the first side chamber to cover the at least one first opening and at least one first side opening, wherein each first membrane includes a first size exclusion cutoff;
a second side chamber formed by a second side chamber body having a second open side that is configured to couple with the second side of the center chamber, the second open side having at least one second side opening configured to fluidly couple with the center chamber through the at least one second opening when the second side chamber is mounted to the center chamber; and
at least one second membrane configured to be positioned between the second side of the center chamber and second open side of the second side chamber to cover the at least one second opening and at least one second side opening, wherein the second membrane includes a second size exclusion cutoff,
wherein the center chamber, first side chamber, and second side chamber are configured to be modular for combining with the first membrane and second membrane in a lateral arrangement.

2. The device of claim 1, wherein the center chamber, first side chamber, and second side chamber are coupled together and combined with the first membrane and second membrane in the lateral arrangement, optionally the first side chamber and/or second side chamber includes an absorbing medium.

3. The device of claim 1, wherein the matrix material includes:
a hydrophilic polysaccharide material, optionally a glycosaminoglycan (GAG), optionally a negatively charged polysaccharide material, optionally hyaluronic acid or hyaluronate; or
a hydrophobic material within the hydrophilic polysaccharide material, wherein the hydrophobic material is optionally a lipid, optionally a lecithin.

4. The device of claim 1, the center chamber body includes one of:
the first side includes one first opening and a second side includes two second openings that are spaced apart from each other, wherein the two second openings have a combined open area that is smaller than an open area of the one first opening; or
the first side includes one first opening and a second side includes one second opening, wherein the one second opening has an open area that is equal to or smaller than an open area of the one first opening.

5. The device of claim 4, wherein the combined open area of the two second openings is less than 90%, 80%, 70%, 60%, 50%, 40%, 30%, 20%, 10%, 5%, or 1% of the open area of the at least one first opening.

6. The device of claim 1, comprising a plurality of different center chamber bodies, each center chamber body having a unique open area of the at least one second side opening.

7. A kit comprising:
the device of claim 1, comprising at least one of:
a plurality of different center chamber bodies;
a plurality of different matrix materials;
a plurality of different first membranes; or
a plurality of second first membranes.

8. A system comprising:
the device of claim 1; and
at least one fluid circulation system having at least one pump operably coupled with at least one of the center chamber, first side chamber, or second side chamber.

9. A method of modeling subcutaneous absorption, comprising:
providing the system of claim 8;
introducing a test agent in a first amount into the matrix material in the center chamber;
allowing the test agent to partition into the first side chamber and/or the second side chamber;
measuring an amount of the test agent in at least one of the: (a) center chamber and/or first side chamber and second side chamber; or (b) both the first and second side chambers;
determining one or more partition parameters regarding absorption of the test agent into the first side chamber and/or second side chamber; and
providing a report having the one or more partition parameters.

10. The method of claim 9, further comprising:
obtaining data of the one or more partition parameters for at least one test agent;
modeling the data with a machine learning system; and
obtaining a machine learning model of the subcutaneous absorption model.

11. The method of claim 9, comprising:
obtaining in vitro subcutaneous data with one or more partition parameters of one or more test agents;
mapping the one or more partition parameters regarding absorption of the test agent with the in vitro subcutaneous data; and
obtaining a correlation model for the subcutaneous absorption model and in vivo data.

12. A computer-implemented method, comprising:
obtaining partition data of a test agent administered to the in vitro device of claim 1, wherein the partition data includes a measured amount of the test agent in at least one of the: (a) center chamber and/or first side chamber and second side chamber; or (b) both the first and second side chambers, wherein the in vitro device emulates subcutaneous absorption and release;
creating input vectors based on the partition data of the test agent;
inputting the input vectors into a machine learning platform;
generating one or more predicted partition parameters regarding absorption of the test agent from the center chamber into the first side chamber and/or second side chamber by the machine learning platform, wherein the one or more predicted partition parameters are specific to test agent in the model; and
preparing a report that includes the one or more predicted partition parameters,
wherein the machine learning platform includes a digital model configured to simulate partition parameters in a subcutaneous model of the in vitro device and the digital model is configured to predict in vivo absorption pharmacokinetic properties of the test agent.

13. One or more non-transitory computer readable media storing instructions that in response to being executed by one or more processors, cause a computer system to perform operations, the operations comprising a computer-implemented method comprising:
obtaining partition data of a test agent administered to the in vitro device of claim 1, wherein the partition data includes a measured amount of the test agent in at least one of the: (a) center chamber and/or first side chamber and second side chamber; or (b) both the first and second side chambers, wherein the in vitro device emulates subcutaneous absorption and release;
creating input vectors based on the partition data of the test agent;
inputting the input vectors into a machine learning platform;
generating one or more predicted partition parameters regarding absorption of the test agent from the center chamber into the first side chamber and/or second side chamber by the machine learning platform, wherein the one or more predicted partition parameters are specific to test agent in the model; and
preparing a report that includes the one or more predicted partition parameters,
wherein the machine learning method performs a Monte Carlo simulation of release of the test agent from the center chamber, and
the partition data can be based on input factors including matrix concentration, test agent injection volume, test agent injection position, and combinations thereof, and
wherein the machine learning platform models a relationship between the input factors and output responses based on a subcutaneous model of the in vitro device.

14. A computer system comprising:
one or more processors; and
one or more non-transitory computer readable media storing instructions that in response to being executed by the one or more processors, cause the computer system to perform operations, the operations comprising a computer-implemented method of:
obtaining partition data of a test agent administered to the in vitro device of claim 1, wherein the partition data includes a measured amount of the test agent in at least one of the: (a) center chamber and/or first side chamber and second side chamber; or (b) both the first and second side chambers, wherein the in vitro device emulates subcutaneous absorption and release;
creating input vectors based on the partition data of the test agent;
inputting the input vectors into a machine learning platform;
generating one or more predicted partition parameters regarding absorption of the test agent from the center chamber into the first side chamber and/or second side chamber by the machine learning platform, wherein the one or more predicted partition parameters are specific to test agent in the model; and
preparing a report that includes the one or more predicted partition parameters,
wherein the partition data can be based on input factors including matrix concentration, test agent injection volume, test agent injection position, and combinations thereof,
wherein the machine learning platform models a relationship between the input factors and output responses based on a subcutaneous model of the in vitro device, and
wherein the machine learning method performs a Monte Carlo simulation of release of the test agent from the center chamber.

15. A computer-implemented method, comprising:
obtaining partition data of a test agent administered to the in vitro device of claim 1, wherein the partition data includes a measured amount of the test agent in at least one of the: (a) center chamber and/or first side chamber and second side chamber; or (b) both the first and second side chambers, wherein the in vitro device emulates subcutaneous absorption and release;
modeling the partition data with a digital model of the subcutaneous model of the in vitro device;
generating one or more predicted partition parameters regarding absorption of the test agent from the center chamber into the first side chamber and/or second side chamber; and
preparing a report that includes the one or more predicted partition parameters.
